(19) United States
(12) Patent Application Publication
(10) Pub. No.: US 2024/0165269 A1
(43) Pub. Date: May 23, 2024

(54) METHODS AND COMPOSITIONS FOR REJUVENATING CNS GLIAL POPULATIONS WITH BCL11A TRANSCRIPTION FACTOR EXPRESSION

(71) Applicant: UNIVERSITY OF ROCHESTER, Rochester, NY (US)

(72) Inventors: Steven A. GOLDMAN, Webster, NY (US); John MARIANI, Rochester, NY (US); Abdellatif BENRAISS, Rochester, NY (US); Benjamin MANSKY, Rochester, NY (US)

(73) Assignee: UNIVERSITY OF ROCHESTER, Rochester, NY (US)

(21) Appl. No.: 18/487,740

(22) Filed: Oct. 16, 2023

Related U.S. Application Data
(60) Provisional application No. 63/380,093, filed on Oct. 19, 2022.

(51) Int. Cl. A61K 48/00; A61K 38/17; A61P 25/28; C12N 15/86

(52) U.S. Cl. CPC A61K 48/0066; A61K 38/1709; A61K 48/0033; A61P 25/28; C12N 15/86; C12N 2740/15043; C12N 2750/14143; C12N 2830/003; C12N 2830/008

(57) ABSTRACT

The present disclosure relates to methods for inducing rejuvenation in a population of glial progenitor cells and methods for treating a subject having a glial cell-related disorder. Methods include administering, to the population of adult glial progenitor cells, an effective amount of an expression vector comprising a nucleotide sequence encoding B-cell lymphoma/leukemia 11A (BCL11A) and a regulatory element operably linked to the nucleotide sequence.

This application claims priority from U.S. Provisional Patent Application No. 63/380,093, filed on Oct. 19, 2022, which is incorporated herein by reference.

This invention was made with government support under NS110776 and AG072298 awarded by National Institutes of Health. The government has certain rights in the invention.

### FIELD

This application relates to methods and compositions for rejuvenating CNS glial populations with an agent expressing a B-cell lymphoma/leukemia 11A (BCL11A) transcription factor.

### BACKGROUND

Glial progenitor cells (GPCs, also referred to as oligodendrocyte progenitor cells and NG2 cells) colonize the human brain during development and persist in abundance throughout adulthood. During development, human GPCs (hGPCs) are highly proliferative bipotential cells, producing new oligodendrocytes and astrocytes. In rodents, this capacity wanes during normal aging, with proliferation, migration, and differentiation competence all diminishing in aged GPCs. Similarly, it was found that adult human GPCs are less proliferative, less migratory, and more readily differentiated than their fetal counterparts when transplanted into congenitally dysmyelinated murine hosts. Yet despite the manifestly different competencies of fetal and adult hGPCs, and the abundant data on GPC transcription in rodent models of aging, little data are available that address changes in GPC gene expression during human aging, or that provide clear head-to-head comparisons of transcription by fetal and adult human GPCs. The present disclosure is directed to overcoming deficiencies in the art.

### SUMMARY

One aspect of the present disclosure relates to a method of inducing rejuvenation in a population of adult glial progenitor cells. The method comprises the step of administering, to the population of adult glial progenitor cells, an effective amount of an expression vector comprising a nucleotide sequence encoding BCL11A and a regulatory element operably linked to the nucleotide sequence.

Another aspect of the present disclosure relates to a method of treating a subject having a glial cell-related disorder. The method comprises the step of administering, to a population of adult glial progenitor cells of the subject, an effective amount of an expression vector comprising a nucleotide sequence encoding BCL11A and a regulatory element operably linked to the nucleotide sequence.

Another aspect of the present disclosure relates to an expression vector that comprises a nucleic acid sequence encoding BCL11A; and a regulatory element operably linked to the nucleic acid sequence.

Another aspect of the present disclosure relates to an adult human glial progenitor cell harboring the expression vector of the present application.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows BCL11A expression is linked to hGPC proliferation. Panel A) Expression of BCL11A by human glial progenitor cells (GPCs) relative to undifferentiated embryonic stem cells (ESCs), as a function of days in vitro (DIV), by RT-qPCR. BCL11A expression decreases during neural induction, increases during GPC specification, and falls again with cell age. Top; timeline defining stages of differentiation. Panel B) Left; representative images of immunostaining for DAPI (blue) and MKI67 (green) in iPSC-derived hGPCs at 100 and 300 DIV. Right, quantification of MKI67+ cells over all DAPI+ cells. N=2. Panel C) Schematic illustrating the design of the CBh-EGFP control virus (top) or CBh-BCL11A-T2A-EGFP. Panel D) Relative proportion of BCL11A+ (left) and MKI67+ cells in the GFP+ and GFP− fraction of LV-BCL11A-transduced hGPCs at DIV 170, normalized to the LV-EGFP condition. Data are presented as means±SEM (N=3). BCL11A is significantly overexpressed in the LV-BCL11A-infected cultures, including by the GFP+ cells (by 31.4±2.8 fold, ** P=0.008, one-sample t-test), and surprisingly, with an upward trend in the GFP− cells as well (8.62±2.66, one-sample t-test P=0.103). More MKI67+ cells were found among the GFP+ cells (2.85±0.61-fold higher than controls, paired t-test *P=0.015) and GFP− cells (2.15±0.42, *P=0.017, paired t-test) in BCL11A-LV cultures. Panel E) RT-qPCR results for BCL11A, MKI67, CDKN1A, CDKN2A, IL1A, and IL8 following treatment of DIV 170 hGPCs with etoposide and either LV-BCL11A or LV-EGFP, normalized to DMSO (N=2-4 per target). BCL11A and MKI67 are downregulated and CDKN1A is increased following etoposide treatment. Restoring BCL11A expression with LV-BCL11A results in a significant decrease in CDKN1A (paired t-test, *** P<0.001), along with a reduction in markers of inflammation and senescence.

FIG. 2 shows BCL11A overexpression induces a proliferative, migratory profile. Panel A) Volcano plot comparing differentially expressed genes in hGPCs one week after transduction with BCL11A or EGFP control. Panel B) Venn diagram showing relative overlap between BCL11A-induced transcripts and those enriched in primary fetal or adult hGPC isolates. Panel C) Heatmap of TPMs for some of the top differentially expressed genes in BCL11A or control cultures. Those highlighted in blue are both downregulated in the BCL11A condition and enriched in adult primary samples, and those in red are both upregulated by BCL11A overexpression and fetal-enriched. Panel D) Upstream regulator analysis from Ingenuity Pathway Analysis (IPA), plotted from highest activation to highest repression. Predicted regulators include genes associated with migration, proliferation, and chromatin regulation. Panel E) Significantly activated and repressed pathways determined via IPA, showing reduction of apoptotic and neural induction pathways, and an increase in proliferative and gliogenic pathways.

FIG. 3 shows Single-cell RNA-seq of BCL11A overexpressing cultures. Panel A) UMAP plots showing cells from 2 sets of BCL11A (top) and GFP (bottom) hGPC cultures. Panel B) Dot plot showing markers of the cell types represented in vitro. Panel C) Violin plots illustrating genes significantly enriched in hGPCs treated with BCL11A (top) vs. their GFP-only controls (bottom). Panel D) UMAP plots colored by the ratio between fetal and adult AUC enrichment score. Panel E) Fetal (left) or adult (right) AUC score by Seurat cluster. Panel F) Comparison of the overall AUC scores in BCL11A and GFP hGPCs. Panel G) Proportion of BCL11A and GFP-treated cells in the most fetal-like and most adult-like clusters.

FIG. 4 shows Mobilization of aged hGPCs in vivo by BCL11A activation. Panel A) Schematic illustrating the design of the RFP tag used in the hGPCs for mouse engraftment. Panel B) Design of the experiment. After 160 days in vitro, RFP+ hGPCs were engrafted into P1 mice. After 100 weeks, animals were injected with LV-EGFP and LV-BCL11A-EGFP, one virus in each hemisphere to serve as an internal control. Three weeks later, mice were harvested for downstream processing. Panel C) Coronal images showing BCL11A overexpression in the BCL11A (top) or GFP (bottom)-infected hemisphere. The dashed line delineates the border of the corpus callosum (CC). Ctx—cortex, Str—striatum. Panel D) Coronal images showing MKI67+ cells in the BCL11A (left) and EGFP infected CC. Panel E) Quantification of BCL11A+ and MKI67+ cells per μm2 in BCL11A or EGFP hemispheres (for BCL11A, LV-BCL11A cells/μm2=4.56e-5±8.23e-6, LV-EGFP cells/μm2=5.06e-6±1.45e-6, paired t-test *P=0.031; for MKI67, LV-BCL11A cells/μm2=1.6e-5±4.06e-6, LV-EGFP cells/μm2=6.63e-6±1.26e-6, paired t-test P=0.079, N=3). Panel F) Coronal images of the chimerized CC. Human cells are marked with RFP and human nuclear antigen (hNA) in magenta, with OLIG2 stained in green. Panel G) Coronal images of the chimerized CC. Human cells are marked with RFP and human nuclear antigen (hNA; magenta), with PDGFRa (green).

FIG. 5 shows BCL11A potentiates a permissive chromatin state at genes for cell migration and survival. Panel A) UMAP plots of human cells isolated from 3 chimerized 2-year-old mouse brains. Bottom, grouped by treatment condition. The dashed line illustrates the cluster unique to the BCL11A condition. Panel B) Plots illustrating the relative expression of markers of the glial lineage, including astrocytes, GPCs, oligodendrocyte-committed GPCs, and oligodendrocytes. Panel C) Violin plots illustrating the transcripts enriched in BCL11A-treated cluster 8 (top), or its counterpart in the EGFP-treated condition. Panel D) UMAP plot of scATAC-seq of hGPC cultures expressing BCL11A or EGFP control. Panel E) Differentially accessible loci in BCL11A (left) or EGFP (right) cells. Panel F) Relative amount of K4me3, K27me3, and K9me3 signal detected genome-wide in BCL11A-overexpressing hGPCs, relative to control (N=2). Panel G) Volcano plots highlighting loci differentially enriched for K4me3 (left), K27me3 (center), and K9me3 (right) in BCL11A or EGFP cells. Panel H) Representative tracks highlighting differences in chromatin mark enrichment at CTNNB1, TEAD2, and PIEZO1.

### DETAILED DESCRIPTION

Herein incorporated by reference is the sequence listing filed with the USPTO named as “1134-138 US.xml” which was created on Oct. 16, 2023, and the size of the XML file is 6,585 in bytes.

Reference will be made in detail to certain aspects and exemplary embodiments of the application, illustrating examples in the accompanying structures and figures. The aspects of the application will be described in conjunction with the exemplary embodiments, including methods, materials and examples, such description is non-limiting, and the scope of the application is intended to encompass all equivalents, alternatives, and modifications, either generally known, or incorporated here. The described aspects, features, advantages, and characteristics of the invention may be combined in any suitable manner in one or more further embodiments. One skilled in the relevant art will recognize that the invention may be practiced without one or more of the specific aspects or advantages of a particular embodiment. In other instances, additional aspects, features, and advantages may be recognized and claimed in certain embodiments that may not be present in all embodiments of the invention. Further, one skilled in the art will recognize many techniques and materials similar or equivalent to those described here, which could be used in the practice of the aspects and embodiments of the present application. The described aspects and embodiments of the application are not limited to the methods and materials described.

Unless otherwise defined, all technical and scientific terms used herein have the same meaning as commonly understood by one of ordinary skill in the art to which this application belongs.

### I. Definitions

As used herein, the following terms or phrases (in parentheses) shall have the following meanings:

As used in this specification and the appended claims, the singular forms “a,” “an” and “the” include plural referents unless the content clearly dictates otherwise. Thus, for example, reference to “a peptide” includes “one or more” peptides or a “plurality” of such peptides.

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

The term “and/or” as used herein means that the listed items are present, or used, individually or in combination. In effect, this term means that “at least one of” or “one or more” of the listed items is used or present.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, and so on. As a non-limiting example, each range discussed herein can be readily broken down into a lower third, middle third and upper third, and so on. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “involving”, “having”, and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps. In embodiments or claims where the term comprising (or the like) is used as the transition phrase, such embodiments can also be envisioned with replacement of the term “comprising” with the terms “consisting of” or “consisting essentially of.” The methods, kits, systems, and/or compositions of the present disclosure can comprise, consist essentially of, or consist of, the components disclosed.

In embodiments comprising an “additional” or “second” component, the second component as used herein is different from the other components or first component. A “third” component is different from the other, first, and second components, and further enumerated or “additional” components are similarly different.

The term “complementary” when used in connection with nucleic acid, refers to the pairing of bases, A with T or U, and G with C. The term “complementary” refers to nucleic acid molecules that are completely complementary, that is, form A to T or U pairs and G to C pairs across the entire reference sequence, as well as molecules that are partially (e.g., at least 80%, 81%, 82%, 83%, 84%, 85%, 86%, 87%, 88%, 89%, 90%, 91%, 92%, 93%, 94%, 95%, 96%, 97%, 98%, or 99%) complementary.

The terms “nucleic acid”, “nucleotide”, and “polynucleotide” encompass both DNA and RNA unless specified otherwise.

The term “polypeptide,” “peptide” or “protein” are used interchangeably and to refer to a polymer of amino acid residues. The terms encompass all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.).

The terms “abrogate”, “abrogation” “eliminate”, or “elimination” of expression of a gene or gene product (e.g., RNA or protein) refers to a complete loss of the transcription and/or translation of a gene or a complete loss of the gene product (e.g., RNA or protein). Expression of a gene or gene product (e.g., RNA or protein) can be detected by standard art known methods such as those described herein, as compared to a control, e.g., an unmodified cell.

The terms “express” and “expression” mean allowing or causing the information in a gene or DNA sequence to become produced, for example producing an RNA or a protein by activating the cellular functions involved in transcription and/or translation of a corresponding gene or DNA sequence. A DNA sequence is expressed in or by a cell to form an “expression product” such as an RNA or a protein. The expression product itself, e.g., the resulting protein, may also be said to be “expressed” by the cell. An expression product can be characterized as intracellular, extracellular or transmembrane

As used herein, the term “glial cells” refers to a population of non-neuronal cells that provide support and nutrition, maintain homeostasis, either form myelin or promote myelination, and participate in signal transmission in the nervous system. “Glial cells” as used herein encompasses fully differentiated cells of the glial lineage, such as oligodendrocytes or astrocytes. For the purpose of the present application/disclosure, the term “glial cells” also refers to glial progenitor cells of various growth or differentiation stages. Each of the glial cells defined in this paragraph can be referred to as macroglial cells.

As used herein, the term “adult glial progenitor cells” refers to glial progenitor cells that are present in a mammal at any developmental stage after birth. In some embodiments, the term “adult glial progenitor cells” refers to glial progenitor cells present in a human subject who is 1, 2, 3, 4, 5, 6, 7, 8, 9, 10, 11, 12, 13, 14, 15, 16, 17, or 18 years of age or older. In some embodiments, the term “adult glial progenitor cells” refers to glial progenitor cells present in a human subject who is 20 years of age or older, 25 years of age or older, 30 years of age or older, 35 years of age or older, 40 years of age or older, 45 years of age or older, or 50 years of age or older. In some embodiments, the term “adult glial progenitor cells” refers to glial progenitor cells present in a human subject of advanced age, such as an adult of 55 years of age or older, 60 years of age or older, 65 years of age or older, 70 years of age or older, 75 years of age or older, or 80 years of age or older.

The term “a functional variant” of a gene product (e.g., a protein), refers to a modified gene product (e.g., by deletion, substitution, insertion, glycosylation, etc.) that retains at least 50% of the biological activity of the unmodified (wild-type) gene product in a competition assay.

The term “effective amount” refers to the amount of active compound or pharmaceutical agent that elicits the biological or medicinal response in a tissue, system, animal, individual or human that is being sought by a researcher, veterinarian, medical doctor or other clinician.

The term “regulatory sequence” or “regulatory element” refers to the nucleic acid sequences or elements that control, regulate, cause or permit expression of a gene to be regulated by such regulatory sequence or element. Regulatory elements/sequences may be found at the 5′ or 3′ side of the coding region, or within the coding region, or within introns, of the gene to be regulated. Examples of regulatory sequences/elements include, but are not limited to, promoters, enhancers, RNA polymerase initiation sites, ribosome binding sites, and other sequences that facilitate the expression of encoded polypeptides in a given expression system

The term “promoter”, as used herein, refers to a nucleotide sequence capable of controlling the expression of a coding sequence or functional RNA. In general, the polynucleotide of interest is located 3′ of a promoter sequence. In some embodiments, the promoter is derived in its entirety from a native gene. In some embodiments, the promoter is composed of different elements derived from different naturally occurring promoters. In some embodiments, the promoter comprises a synthetic nucleotide sequence. It will be understood by those skilled in the art that different promoters will direct the expression of a gene in different tissues or cell types, or at different stages of development, or in response to different environmental conditions or to the presence or the absence of a drug or transcriptional co-factor. Ubiquitous, cell-type-specific, tissue-specific, developmental stage-specific, and conditional promoters, for example, drug-responsive promoters (e.g. tetracycline-responsive promoters) are well known to those of skill in the art. Examples of promoter include, but are not limited to, the phophoglycerate kinase (PKG) promoter, CAG, NSE (neuronal specific enolase), synapsin or NeuN promoters, the SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), SFFV promoter, rous sarcoma virus (RSV) promoter, synthetic promoters, hybrid promoters, and the like. The promoters can be of human origin or from other species, including from mice. In addition, sequences derived from nonviral genes, such as the murine metallothionein gene promoter, will also find use herein. In some embodiments, the promoter is a heterologous promoter. In some embodiments, a promoter sequence consists of proximal and more distal upstream elements and can comprise an enhancer element.

The term “heterologous promoter”, as used herein, refers to a promoter that does is not found to be operatively linked to a given encoding sequence in nature.

The term “enhancer” refers to a nucleotide sequence that can stimulate promoter activity and may be an innate element of the promoter or a heterologous element inserted to enhance the level or tissue-specificity of a promoter.

The term “operatively linked” or “operably linked” refers to the association of two or more nucleic acid fragments on a single nucleic acid fragment so that the function of one is affected by the other. For example, a promoter is operatively linked with a coding sequence when it is capable of affecting the expression of that coding sequence (e.g., the coding sequence is under the transcriptional control of the promoter). Encoding sequences can be operatively linked to regulatory sequences in sense or antisense orientation.

The term “transcription factor” refers to a DNA-binding protein that regulate the expression of specific genes. A transcription factor can have a positive effect on gene transcription and, thus, may be referred to as an “activator” or a “transcriptional activation factor.” A transcription factor can also negatively affect gene expression and, thus, may be referred to as “repressor” or a “transcription repression factor.”

A process of rejuvenation is observed when one or all of these markers of aging phenotype is reduced or suppressed in an aged or senescent cell type due to the rejuvenating process.

Certain terms employed in the specification, examples, and claims are collected herein. Unless defined otherwise, all technical and scientific terms used in this disclosure have the same meanings as commonly understood by one of ordinary skill in the art to which this disclosure belongs.

Preferences and options for a given aspect, feature, embodiment, or parameter of the disclosure should, unless the context indicates otherwise, be regarded as having been disclosed in combination with any and all preferences and options for all other aspects, features, embodiments, and parameters of the disclosure.

### II. Methods Involving the Promotion of Expression and/or Activity of BCL11A Transcription Factor

A. Method of Inducing Rejuvenation in a Population of Glial Progenitor Cells

One aspect of the present disclosure relates to a method of inducing rejuvenation in a population of glial progenitor cells. In some embodiments, the method comprises promoting the activity and/or enhancing expression of B-cell lymphoma/leukemia 11A (BCL11A) in the population of glial progenitor cells at levels that induce rejuvenation of the glial progenitor cells.

The BCL11A is a transcription factor that is expressed in the brain and forms a protein complex with CASK to regulate axon outgrowth and branching. Furthermore, BCL11A binds to the TBR1 regulatory region and inhibits the expression of TBR1, a neuron-specific protein that is implicated in intellectual disability and autism spectrum disorder.

Human BCL11A is encoded by a gene located on chromosome 2p16.1 and highly conserved to mouse BCL11A (musBcl11a). It encodes a 125 kDa Kruppel-like zinc-finger protein containing six C2H2 zinc-fingers, a proline-rich region, and an acidic domain. BCL11A specifically binds to 5′-GGCCGG-3′ sequences and functions mainly as a transcriptional repressor. BCL11A is mainly expressed in brain and most hematopoietic cells, including hematopoietic stem cells, common lymphoid progenitors, B cells, and early T-cell progenitors, although it is weakly expressed in T lymphocytes. As shown in Table 1, human BCL11A has a number of spliced transcript variants.

TABLE 1
Exemplary Human Genes and Transcript Variants

| | | | NCBI Reference |
|---|---|---|---|
| | Gene | | Transcript |
| Gene | ID No.* | Transcript Variant | Accession Nos.* |
| BAF chromatin | 53335 | transcript variant 3 | NM_138559.2 |
| remodeling | | transcript variant 2 | NM_018014.4 |
| complex | | transcript variant 4 | NM_001363864.1 |
| subunit | | transcript variant X3 | XM_011532910.1 |
| (BCL11A) | | transcript variant X1 | XM_011532909.1 |
| | | transcript variant 1 | NM_022893.4 |
| | | transcript variant 5 | NM_001365609.1 |
| | | transcript variant X7 | XM_024452962.1 |
| | | transcript variant X5 | XM_017004335.1 |
| | | transcript variant X2 | XM_017004333.1 |
| | | transcript variant X7 | XM_024452963.1 |
| | | transcript variant X8 | XM_017004336.1 |

*Each of which is hereby incorporated by reference in its entirety.

In some embodiments, the method of the present application comprises the step of expressing, in the population of glial progenitor cells, an effective amount of BCL11A protein. In some embodiments, the glial progenitor cells are adult glial progenitor cells.

In some embodiments, the step of expressing comprises administering, to the population of glial progenitor or adult glial progenitor cells, an effective amount of an expression vector that is capable of expressing BCL11A in the glial progenitor or adult glial progenitor cells in an effective amount to induce rejuvenation of the glial progenitor or adult glial cells. In some embodiments, the expression vector comprises a nucleotide sequence encoding BCL11A and a regulatory element operably linked to the nucleotide sequence. In some embodiments, the administering is carried out ex vivo. In some embodiments, the administering is carried out in vivo.

The term “rejuvenating” or “rejuvenation”, when used in the context of glial or adult glial cells or glial progenitor or adult glial progenitor cells, refers to a reversion of the aging process in said cells and a return to youthful cell state, in particular with regard to proliferative and/or differentiation capacity without loss of cell identity.

The cellular aging phenotype in the above said glial or glial progenitor cells can be characterized, inter alia, by the following markers: (1) increased expression of one or more markers of senescence, such as CDKN1A, CDKN2A, E2F6, ZNF274, IKZF3, and ILIA, or markers of glial differentiation, such as BCAS1, CLDN11, CNP, LPAR1, MAG, OGT, PLP1, PMP22, and MYRF and (2) decreased expression of one or more youth markers associated with cellular youth, such as BCAN, CCND2, CDK1, CDK4, CDK5, CENPF, CHEK1, CHRDL1, FN1, HMGA2, LMNB1, MKI67, MYC, NFIB, TEAD1, TEAD2, and YAP1, and of glial ontogeny in particular, including PCDH15, PDGFRA, PTPRZ1, ST8SIA1, and CSPG4.

As used in this application, a process of rejuvenation is observed when (1) expression of one or more of the senescence markers is reduced and/or (2) expression of one or more of the youth markers is enhanced. In some embodiments, a process of rejuvenation is observed when increased expression of one or more youth markers consistent with reacquisition mitotic and differentiation competence is observed. Examples of youth markers consistent with reacquisition mitotic and differentiation competence include, but are not limited to CCND2, CDK1, CDK4, CDK5, CENPF, CHEK1, FN1, HMGA2, LMNB1, MKI67, MYC, NFIB, PATZ1, TEAD1, TEAD2, TP53 and YAP1.

In some embodiments, a process of rejuvenation is observed when increased expression of one or more youth markers consistent with functional glial progenitor cell state is observed. Examples of youth markers consistent with functional glial progenitor cell state include, but are not limited to BCAN, CA10, CHRDL1, CSPG4, NXPH1, PCDH15, PDGFRA, PTPRZ1 and ST8SIA1.

In some embodiments, an agent has a rejuvenating effect on (1) a glial or adult glial cell or the cell population thereof; or (2) a glial progenitor or adult glial progenitor cell, or the cell population thereof, if the expression of one or more youth markers is significantly increased in the above said cells or cell populations after treatment with the agent.

In some embodiments, an agent has a rejuvenating effect on (1) a glial or adult glial cell or the cell population thereof; or (2) a glial progenitor or adult glial progenitor cell, or the cell population thereof, if the expression of one or more senescence markers is significantly decreased in the above said cells or cell populations after treatment with the agent.

In some embodiments, an agent has a rejuvenating effect on (1) a glial or adult glial cell or the cell population thereof; or (2) a glial progenitor or adult glial progenitor cell, or the cell population thereof, if the expression of one or more youth marker is significantly increased, and the expression of one or more senescence marker is significantly decreased in the above said cells or the cell populations after treatment with the agent.

As used herein, the expression of a gene expression is “significantly increased,” if the gene expression is increased by 30% or greater, 50% or greater, 100% or greater, 150% or greater, 200% or greater, 300% or greater, 400% or greater, 500% or greater, 600% or greater, 700% or greater, 800% or greater, 900% or greater, or 1000% or greater at the mRNA level; or is increased by 10% or greater, 20% or greater, 30% or greater, 40% or greater, 50% or greater, 60% or greater, 70% or greater, 80% or greater, 90% or greater, or 100% or greater at the protein level.

As used herein, the expression of a gene is “significantly decreased” if the gene expression is decreased by 30% or greater, 50% or greater, 60% or greater, 70% or greater, 80% or greater, or 90% or greater at the mRNA level, or decreased by 10% or greater, 20% or greater, 30% or greater, 40% or greater, 50% or greater, 60% or greater, 70% or greater, 80% or greater, or 90% or greater at the protein level.

In some embodiments, the method of the present application comprises the step of expressing, in the population of glial progenitor cells, an effective amount of BCL11A protein that results in an increased expression of MKI67 gene in the glial progenitor cells.

In some embodiments, the method of the present application comprises the step of expressing, in the population of glial progenitor cells, an effective amount of BCL11A protein that results in activation of aged glial progenitor cells. In some embodiments, the activation of aged glial progenitor cells is evidenced by mitotic expansion and/or migratory colonization of the aged glial progenitor cells.

In some embodiments, the method of the present application comprises the step of expressing, in the population of glial progenitor cells, an effective amount of BCL11A protein that results in the mobilization of, and remyelination by, aged glial progenitor cells. One skilled in the art would recognize that remyelination by such previously aged progenitor cells would result in therapeutic benefits in treating glial cell-related disorders or conditions.

Glial progenitor cells suitable for use in the methods disclosed herein include mammalian glial progenitor cells, e.g., human glial progenitor cells, rodent glial progenitor cells, non-human primate glial progenitor cells, ovine glial progenitor cells, bovine glial progenitor cells, porcine glial progenitor cells, canine glial progenitor cells, and feline glial progenitor cells. In some embodiments, the adult glial progenitor cells are adult human glial progenitor cells.

In some embodiments, the expression vector comprises a nucleotide sequence encoding human BCL11A or a functional variant thereof. In some embodiments, the expression vector comprises a nucleotide sequence encoding human BCL11A. In some embodiments, the expression vector comprises a nucleotide sequence encoding SEQ ID NO:2.

In some embodiments, the regulatory element comprises a ubiquitous promoter, such as chicken beta-actin (CBA) promoter, hybrid form of the CBA promoter (CBh) CAG promoter, cytomegalovirus (CMV) promoter and rous sarcoma virus (RSV) promoter.

In some embodiments, the regulatory element comprises a promoter and/or enhance of a gene which is selectively expressed by glial progenitor cells, such as the promoter/enhancer of platelet derived growth factor alpha (PDGFRA), zinc finger protein 488 (ZNF488), G protein-coupled receptor (GPR17), oligodendrocyte Transcription Factor 2 (OLIG2), chondroitin sulfate proteoglycan 4 (CSPG4), and SRY-box transcription factor 10 (SOX10).

In some embodiments, the regulatory element comprises an inducible promoter. An inducible promoter is capable of directly or indirectly activating transcription of the nucleic acid molecule that it is operatively coupled to in response to a “regulatory agent” (e.g., a chemical agent or biological molecule, such as a metabolite, a small molecule) or a stimulus. In the absence of a “regulatory agent” or stimulus, the nucleotide sequence encoding BCL11A will not be transcribed. The term “not transcribed” or “not substantially expressed” means that the level of transcription is at least 10-fold, 20-fold, 30-fold, 40-fold, or 50-fold lower than the level of transcription observed in the presence of an appropriate stimulus or regulatory agent; and preferably at least 100-fold, 250-fold, or 500-fold or lower than the level of transcription observed in the presence of an appropriate stimulus or regulatory agent.

Inducible promoters suitable for use in the expression vector of the present application include, but are not limited to, those regulated by hormones and hormone analogs such as progesterone, ecdysone and glucocorticoids as well as promoters which are regulated by tetracycline, heat shock, heavy metal ions, interferon, and lactose operon activating compounds. For a review of these systems see Gingrich & Roder, “Inducible Gene Expression in the Nervous System of Transgenic Mice,” Annu. Rev. Neurosci. 21:377-405 (1998), which is hereby incorporated by reference in its entirety. Tissue-specific expression has been well characterized in the field of gene expression and tissue-specific and other inducible promoters are well known in the art.

In some embodiments, the regulatory element of the expression vector of the present application comprises an inducible promoter. When rejuvenation of the glial progenitor cells is desired, it is achieved by administering a suitable regulatory agent (e.g., doxycycline, hormone) or other inducing agent to the subject, preferably using a route or other means that is targeted to the glial progenitor cells harboring the expression vector. Contacting glial progenitor cells harboring the expression vector with a regulatory agent induces expression of the system designed to express BCL11A. However, it should be recognized by one skilled in the art that in other inducible vectors, the opposite is true, that is, the regulatory agent inhibits expression and removal permits expression. Suitable inducible promoter for inclusion in the systems of the present disclosure are well known in the art and include, without limitation, a tetracycline-controlled operator system, a cumate-controlled operator system, a rapamycin inducible system, a FKCsA inducible system, and an ABA inducible system (see, e.g., Kallunki et al., “How to Choose the Right Inducible Gene Expression System for Mammalian Studies?” Cells 8(8):796 (2019); U.S. Pat. Nos. 8,728,759; and 7,745,592, which are hereby incorporated by reference in their entirety).

In some embodiments, the expression vector of the present application comprises a tetracycline-controlled operator system (tet-on promoter), wherein transcription of the gene of interest is activated in the presence of tetracycline. In some embodiments, the expression vector of the present application comprises a tetracycline-controlled operator system (Tet-off promoter), wherein transcription of the gene of interest is activated in the absence of tetracycline.

In some embodiments, the expression vector of the present application comprises a cumate-controlled operator system, wherein transcription of the gene of interest is activated in the presence of cumate. In some embodiments, the expression vector of the present application comprises a cumate-controlled operator system, wherein transcription of the gene of interest is activated in the absence of cumate.

In some embodiments, the expression vector of the present application comprises a rapamycin-controlled operator system, wherein transcription of the gene of interest is activated in the presence of rapamycin.

In some embodiments, the expression vector of the present application is a non-viral vector. In some embodiments, the non-viral vector is a plasmid.

In some embodiments, the expression vector of the present application is a viral vector. Examples of viral vectors include, but are not limited to, adenovirus vectors, adeno-associated virus (“AAV”) vectors, retrovirus vectors, lentivirus vectors (i.e., a lentiviral vector), vaccinia virus vectors, herpes virus vectors, and any other viral vectors suitable for introduction of the gene of interest (e.g., BCL11A) described herein into a given organism or genetic background by any means to facilitate expression of the gene of interest.

In some embodiments, the expression vector of the present application vector is a lentiviral vector.

In some embodiments, the expression vector of the present application vector is an AAV vector.

In some embodiments, the expression vector of the present application vector is a retroviral vector.

Methods for generating and isolating viral expression vectors suitable for use as expression vectors are known in the art.

In some embodiments, the step of expressing comprises administering, to the population of glial progenitor cells, an effective amount of an expression vector that is capable of expressing an agent that inhibits the activity of an endogenous repressor of BCL11A activity in the adult glial progenitor cells. Examples of repressor of BCL11A activity include, but are not limited to, KLF1, POGZ, HRI, Mi2β, SOX2 and FOXQ1.

In some embodiments, the method of the present application further comprises the step of promoting expression of one or more rejuvenation-promoting genes in the population of glial progenitor cells. As used herein, the term “rejuvenation-promoting genes” refers to genes that contribute to reversing, slowing aging, leading to younger cells, or tissues.

In some embodiments, the one or more additional rejuvenation-promoting genes are selected from the group consisting of ARX, CEBPZ, DLX1, DLX2, ELK1, ETS1, ETV4, KLF16, MYBL2, MYC, NFYB, POU3F1, SMAD1, SOX3, SP5, TCF12, TFDP1, TP53, ZIC3, and ZNF195.

In some embodiments, the one or more additional rejuvenation-promoting genes are selected from the group consisting of CEBPZ, MYBL2, MYC, NFYB and ZNF195.

In some embodiments, the one or more additional rejuvenation-promoting genes are selected from the group consisting of ACTB, AKR1C1, ANAPC11, AP2B1, APLP2, APOD, ARF5, ARL4A, ARPC3, ARPP19, ATOX1, ATP5F1E, ATP5MC1, ATP5MC3, ATP5MD, ATP5ME, ATP5MF, ATP5MG, ATP5MPL, ATP5PF, ATP6V0B, ATP6V0E1, ATXN7L3B, B2M, B3GAT2, BEX1, BEX3, BEX5, BLOC1S1, BMERB1, C18orf32, C1orf122, C1QBP, C4orf48, CADM4, CALM1, CALM3, CALR, CANX, CAV2, CC2D1A, CCND1, CCNI, CD63, CD82, CDC42, CDH2, CFL1, CHCHD2, CHGB, CIAO2B, CLCN3, CLTA, CLTC, CNN3, CNTN1, COTL1, COX4I1, COX6A1, COX6C, COX7A2, COX7C, COX8A, CPNE8, CPS1, CRNDE, CSPG4, CTHRC1, CUL4B, CYP51A1, DBI, DCX, DDAH1, DDX1, DENND10, DMD, DMRT2, DNAJA2, DPYSL2, DRAP1, DSTN, DYNC112, EDF1, EDIL3, EEF1A1, EEF1B2, EEF2, EID1, EIF3J, ELOB, EMC10, EMP2, ESD, ETV1, FABP7, FAM171B, FAM177A1, FAU, FIS1, FXYD6, GADD45A, GAP43, GCSH, GNAS, GOLM1, GPM6B, GSTP1, H3-3A, H3-3B, HINT1, HNRNPA1, HNRNPA3, HNRNPAB, HNRNPC, HNRNPK, HNRNPM, HNRNPR, HSPA5, IGFBP2, ITGB8, ITM2A, ITM2B, JPT1, KDELR1, KLRK1-AS1, KRTCAP2, KTN1, LDHB, LHFPL3, LRRC4B, LY6H, MAP2, MARCKS, MARCKSL1, MIA, MICOS10, MIF, MIR9-1HG, MMGT1, MPZL1, MT3, MTLN, MTRNR2L12, MTRNR2L8, MYL12A, MYL12B, NACA, NARS1, NCL, NDUFA1, NDUFA11, NDUFA13, NDUFA3, NDUFA4, NDUFB1, NDUFB11, NDUFB2, NDUFB6, NDUFB7, NDUFC2, NDUFS5, NEU4, NUCKS1, OAZ1, OLFM2, OSBPL8, OST4, OSTC, PABPC1, PCBP2, PCDH10, PCDH11X, PCDH17, PCDHB2, PCDHGB6, PDGFRA, PDIA6, PEBP1, PEG10, PFN1, PGRMC1, PKIA, PLPP3, PLPPR1, PPIA, PRDX1, PRDX2, PRDX5, PSMB1, PSMB9, PTMS, PTN, PTPRA, RAB10, RAB14, RAB2A, RAB31, RAC1, RACK1, RMDN2, RAMP1, RO60, ROBO1, RRAGB, RTN3, S100B, SARAF, SAT1, SBDS, SCARB2, SCP2, SCRG1, SEC62, SELENOK, SELENOT, SELENOW, SERF2, SERPINE2, SET, SH3BGRL, SKP1, SLC25A6, SLIT2, SLITRK2, SMC3, SMDT1, SMOC1, SMS, SNCA, SNHG29, SNHG6, SNX3, SNX22, SOD1, SOX11, SOX2, SOX9, SPCS2, SPCS3, SRP14, SSR4, STAG2, STMN1, SUPT16H, TALDO1, TBCB, TCEAL7, TCEAL8, TCEAL9, TIMP1, TLE5, TM4SF1, TM9SF3, TMA7, TMBIM6, TMCO1, TMEM147, TMEM258, TMEM50A, TMOD2, TMSB10, TMSB4X, TPT1, TRAF4, TRIO, TSC22D4, TSPAN6, TSPAN7, TTC3, TUBB, UBA52, UBL5, UQCR10, UQCR11, UQCRB, VIM, WSB2, WSCD1, YBX1, YWHAB, YWHAE, ZFAS1, ZNF428, and ZNF462.

In some embodiments, the one or more additional rejuvenation-promoting genes are selected from the group consisting of APOD, B2M, BEX3, BEX5, CCND1, CTHRC1, EDIL3, EMC10, FABP7, GADD45A, ITM2A, LRRC4B, LY6H, MIA, MT3, NEU4, OLFM2, PTMS, RAMP1, SNX3, TRAF4, TRIO, UBA52, and YWHAB.

In some embodiments, the one or more additional rejuvenation-promoting genes are selected from the group consisting of ANAPC11, APOD, ATP5MC3, B2M, CALM1, MT3, NEU4, PEBP1, RAMP1, SOD1 and TBCB.

In some embodiments, the one or more additional rejuvenation-promoting genes are selected from the group consisting of APOD, BEX3, BEX5, CCND1, CTHRC1, EDIL3, EMC10, GADD45A, ITM2A, MIA, TRAF4, and TRIO.

In some embodiments, the one or more additional rejuvenation-promoting genes are selected from the group consisting of B2M, FABP7, LRRC4B, LY6H, MT3, NEU4, OLFM2, PTMS, RAMP1, SNX3, UBA52, and YWHAB.

In some embodiments, the one or more additional rejuvenation-promoting genes are selected from the group consisting of LY6H, MIA, GADD45A, ITM2A and ITM2B.

In some embodiments, the one or more additional rejuvenation-promoting genes are selected from the group consisting of HDAC2, EZH2, MYC, HMGA2, NFIB and TEAD2.

In some embodiments, the method of the present application further comprises the step of inhibiting expression of one or more rejuvenation-suppressing genes in the population of glial progenitor cells. As used herein, the term “rejuvenation-suppressing genes” refers to genes that have opposite functions of rejuvenation-promoting genes.

In some embodiments, the one or more rejuvenation-suppressing genes are selected from the group consisting of ABCG1, ADGRB1, ADGRG1, AKAP9, AL360181.3, ANKRD10, ARGLU1, ARL4C, ARL16, ARMCX6, ATP1A2, ATP1B3, ATP10B, B3GNT7, BHLHE41, BPTF, BRI3, BX664615.2, BX890604.1, C1QL2, CAMK2N1, CCDC85B, CCNL1, CHCHD10, CHORDC1, CIRBP, CLDN10, COL9A1, COL9A2, CXADR, DANCR, DCXR, DHX36, DLL3, DNAJA1, DNM3, ECH1, EGR1, EIF1AX, ELAVL3, EMID1, ETFB, FABP5, FAM133A, FAM133B, FBXO2, FERMT1, FIBIN, FOS, FOSB, FSCN1, FSIP2, GABPB1-AS1, GALR1, GNG8, GNPTAB, GOLGA8A, GOLGA8B, GPR155, GRID2, GRM7, HAPLN1, HMX1, HSPA1A, HSPA1B, HSPH1, HTRA1, IGFBP2, JAG1, JUN, JUNB, KCNIP4, KCNQ1OT1, KLF3-AS1, LAMP2, LINC01116, LINC01301, LINC01896, LRP4, LRRC7, MACF1, MALAT1, MAP3K13, MASP1, MDH1, MT1E, MT2A, MYT1, NASP, NKTR, NUTM2A-AS1, OFD1, PCDHB5, PCDHGA3, PCDHGB6, PEPD, PHGDH, PLCG2, PMP2, PNISR, PPP1R14A, PTGDS, RAB3IP, RAF1, RAP1GAP, RARRES2, RBM25, RBMX, REV3L, RHOBTB3, RIMS2, RIT2, RRBP1, RSRP1, S100A1, S100A16, SAT1, SCG2, SEMA3E, SERTAD1, SEZ6L, SEZ6L2, SH3GLB2, SNHG15, SNRNP70, SPARCL1, SRSF5, STAT3, STXBP6, SYNRG, THBS4, TLE4, TMEM176B, TPI1, TSC22D3, USP11, VCAN, WFDC1, WSB1, ZFYVE16, ZNF528, and ZNF528-AS1.

In some embodiments, the one or more rejuvenation-suppressing genes are selected from the group consisting of ADGRG1 ARL4C, ARMCX6, ATP1A2, ATP1B3, B3GNT7, CXADR, DLL3, FABP5, FIBIN, IGFBP2, LRRC7, MAP3K13, MT1E, MT2A, PCDHGA3, PCDHGB6, PLCG2, PTGDS, SAT1, SEZ6L, SPARCL1, THBS4, and TLE4.

In some embodiments, the one or more rejuvenation-suppressing genes are selected from the group consisting of ARGLU1, EGR1, FSIP2, HSPH1, MACF1, NKTR, RBMX, STAT3, TLE4, and WSB1.

In some embodiments, the one or more rejuvenation-suppressing genes are selected from the group consisting of ADGRG1, ATP1A2, ATP1B3, B3GNT7, CXADR, DLL3, FABP5, MT1E, MT2A, PTGDS, SEZ6L, and THBS4.

In some embodiments, the one or more rejuvenation-suppressing genes are selected from the group consisting of ARL4C, ARMCX6, FIBIN, IGFBP2, LRRC7, MAP3K13, PCDHGA3, PCDHGB6, PLCG2, SAT1, SPARCL1, and TLE4.

In some embodiments, the one or more rejuvenation-suppressing genes are selected from the group consisting of ZNF274, MAX, E2F6, IKZF3 and STAT3.

In some embodiments, the step of inhibiting expression of one or more rejuvenation-suppressing genes comprises administering, to the population of glial progenitor cells, an expression vector encoding one or more microRNAs. As use herein, the term “microRNA” or “miRNA” refers to a class of small RNA molecules that may negatively regulate gene expression. In some embodiments, the one or more microRNAs are selected from the group consisting of miR-193a-5P, miR-23b-3-p, miR-4687-3p, miR-4651, miR-4270 and miR-24-3p. May need to expand the group.

In some embodiments, the step of inhibiting expression of one or more rejuvenation-suppressing genes comprises administering, to the population of glial progenitor cells, an expression vector encoding one or more shRNAs. Short hairpin RNA (shRNA) molecules comprise the sense and antisense sequences from a target gene connected by a loop. Once transcribed, shRNA molecules are transported from the nucleus into the cytoplasm where the enzyme Dicer processes them into small/short interfering RNAs (siRNAs). As used herein, the term “short hairpin RNA interference” or “shRNAi” refers to a process that is mediated by a class of small RNA molecules that negatively regulate gene expression.

In some embodiments, the step of inhibiting expression of one or more rejuvenation-suppressing genes comprises administering, to the population of glial progenitor cells, one or more antisense oligonucleotides, or an expression vector encoding one or more antisense oligonucleotides. As used herein, the term “antisense oligonucleotide” or “ASO” refers to small (˜18-30 nucleotides), synthetic, single-stranded nucleic acid polymers of diverse chemistries, which can be employed to modulate gene expression via various mechanisms. ASOs can be subdivided into two major categories: RNase H competent and steric block. The endogenous RNase H enzyme RNASEH1 recognizes RNA-DNA heteroduplex substrates that are formed when DNA-based oligonucleotides bind to their cognate mRNA transcripts and catalyzes the degradation of RNA. Cleavage at the site of ASO binding results in destruction of the target RNA, thereby silencing target gene expression. Steric block oligonucleotides are ASOs that are designed to bind to target transcripts with high affinity but do not induce target transcript degradation as they lack RNase H competence. Such oligonucleotides therefore comprise either nucleotides that do not form RNase H substrates when paired with RNA or a mixture of nucleotide chemistries (that is, ‘mixmers’) such that runs of consecutive DNA-like bases are avoided.

In some embodiments, the step of inhibiting expression of one or more rejuvenation-suppressing genes comprises administering, to the population of glial progenitor cells, an expression vector encoding a nuclease-based gene editing system. As used herein, the term “nuclease-based gene editing system” refers to a system comprising a nuclease or a derivative thereof that can be recruited to a target sequence in the genome. Examples of nuclease-based gene editing systems include, but are not limited to, Clustered Regularly Interspaced Short Palindromic Repeat-associated (“Cas”) protein (e.g., Cas9, Cas12a, and Cas12b) related system (CRISPR-CAS system), zinc finger nuclease-related system (“ZFN system”), and transcription activator-like effector nucleases-related system (“TALEN system”).

B. Method of Treating a Glial Cell-Related Disorder.

Another aspect of the present disclosure relates to a method of treating a subject having a glial cell-related disorder. The method comprises the step of administering, to a population of glial progenitor cells of the subject, an effective amount of an agent that promotes the activity and/or enhances expression of BCL11A in the population of glial progenitor cells.

In some embodiments, the a glial cell-related disorder is selected from the group consisting of multiple sclerosis, neuromyelitis optica, transverse myelitis, optic neuritis, subcortical stroke, diabetic leukoencephalopathy, hypertensive leukoencephalopathy, age-related white matter disease, spinal cord injury, radiation- or chemotherapy induced demyelination, post-infectious and post-vaccinial leukoencephalitis, periventricular leukomalacia, pediatric leukodystrophiey (e.g., Pelizaeus-Merzbacher Disease, Tay-Sach Disease, Sandhoff's gangliosidoses, Krabbe's disease, metachromatic leukodystrophy, mucopolysaccharidoses, Niemann-Pick A disease, adrenoleukodystrophy, Canavan's disease, Vanishing White Matter Disease, and Alexander Disease), lysosomal storage diseases, congenital dysmyelination, inflammatory demyelination, vascular demyelination, and cerebral palsy.

In some embodiments, the glial cell-related disorder is a neurodegenerative disorder selected from the group consisting of Huntington's disease, frontotemporal dementia, Parkinson's disease, multisystem atrophy, and amyotrophic lateral sclerosis.

In some embodiments, the glial cell-related disorder is a neuropsychiatric disorder selected from the group consisting of schizophrenia, autism spectrum disorder, and bipolar disorder.

In some embodiments, the glial cell-related disorder is a myelin disease, wherein the myelin disease is leukodystrophy or a white matter disease.

In some embodiments, “treating” a subject having a glial cell-related disorder encompasses: (1) preventing, delaying, or reducing the incidence and/or likelihood of the appearance of at least one clinical or sub-clinical symptom of the glial cell-related disorder developing in a subject that may be afflicted with or predisposed to the glial cell-related disorder, but does not yet experience or display clinical or subclinical symptoms of the glial cell-related disorder; or (2) inhibiting the glial cell-related disorder, i.e., arresting, reducing or delaying the development of the myelination deficiency or a relapse thereof or at least one clinical or sub-clinical symptom thereof; or (3) relieving the glial cell-related disorder, i.e., causing regression of the glial cell-related disorder or at least one of its clinical or sub-clinical symptoms. The benefit to a subject to be treated is either statistically significant or at least perceptible to the patient or to the physician.

As used herein, the term “subject” refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cat, or a dog. In some embodiments, the subject is a research animal. In some embodiments, the subject is genetically engineered, e.g., a genetically engineered non-human subject.

The subject may be an adult subject. In some embodiments, the subject is at least 1 year old, least 2 year old, least 4 year old, least 6 year old, least 8 year old, least 10 year old, least 12 year old, least 15 year old, at least 18 years old, at least 20 years old, at least 25 years old, at least 30 years old, at least 35 years old, at least 40 years old, at least 45 years old, at least 50 years old, at least 55 years old, at least 60 years old, at least 65 years old, at least 70 years old, at least 75 years old, at least 80 years old, at least 85 years old, at least 90 years old, at least 95 years old, at least 100 years old, or more.

In some embodiments, the subject is an adult subject between 18 to 100 years old, 20 to 100 years old, 30 to 100 years old, 40 to 100 years old, 50 to 100 years old, 50 to 100 years old, 60 to 100 years old, 70 to 100 years old, 80 to 100 years old, or 90 to 100 years old.

In some embodiments, the agent that promotes the activity and/or enhances expression of BCL11A is an agent that expresses an effective amount of BCL11A protein that results in an increased expression of MKI67 gene in the glial progenitor cells.

In some embodiments, the agent that promotes the activity and/or enhances expression of BCL11A is an agent that expresses an effective amount of BCL11A protein that results in activation of aged glial progenitor cells. In some embodiments, the activation of aged glial progenitor cells is evidenced by mitotic expansion and/or migratory colonization of the aged glial progenitor cells.

In some embodiments, the agent that promotes the activity and/or enhances expression of BCL11A is an agent that expresses an effective amount of BCL11A protein that results in remyelination of aged glial cells.

In some embodiments, the agent is an expression vector comprising a nucleotide sequence encoding BCL11A and a regulatory element operably linked to the nucleotide sequence.

In some embodiments, the expression vector comprises a nucleotide sequence encoding human BCL11A or a functional variant thereof. In some embodiments, the expression vector comprises a nucleotide sequence encoding human BCL11A. In some embodiments, the expression vector comprises a nucleotide sequence encoding SEQ ID NO:2.

In some embodiments, the regulatory element comprises a ubiquitous promoter, such as chicken beta-actin (CBA) promoter, hybrid form of the CBA promoter (CBh) CAG promoter, cytomegalovirus (CMV) promoter and rous sarcoma virus (RSV) promoter.

In some embodiments, the regulatory element comprises a promoter and/or enhance of a gene which is selectively expressed by glial progenitor cells, such as the promoter/enhancer of platelet derived growth factor alpha (PDGFRA), zinc finger protein 488 (ZNF488), G protein-coupled receptor (GPR17), oligodendrocyte Transcription Factor 2 (OLIG2), chondroitin sulfate proteoglycan 4 (CSPG4), and SRY-box transcription factor 10 (SOX10).

In some embodiments, the regulatory element comprises an inducible promoter. In some embodiments, the inducible promoter is a tet-on or tetp-off promoter. In some embodiments, the inducible promoter a cumate-controlled operator system, wherein transcription of the gene of interest is activated in the presence of cumate. In some embodiments, the expression vector of the present application comprises a cumate-controlled operator system, wherein transcription of the gene of interest is activated in the absence of cumate. In some embodiments, the inducible promoter comprises a rapamycin-controlled operator system, wherein transcription of the gene of interest is activated in the presence of rapamycin.

In some embodiments, the expression vector is a non-viral vector. In some embodiments, the non-viral vector is a plasmid.

In some embodiments, the expression vector a viral vector. In some embodiments, the viral vector is a lentiviral vector, an AAV vector or a retroviral vector.

In some embodiments, the agent comprises an expression vector that is capable of expressing an agent that inhibits the activity of an endogenous repressor of BCL11A activity in the adult glial progenitor cells. Examples of repressor of BCL11A activity include, but are not limited to, KLF1, POGZ, HRI, Mi2β, SOX2 and FOXQ1.

In some embodiments, the method further comprises the step of promoting expression of one or more rejuvenation-promoting genes in the population of glial progenitor cells.

In some embodiments, the one or more additional rejuvenation-promoting genes are selected from the group consisting of ARX, CEBPZ, DLX1, DLX2, ELK1, ETS1, ETV4, KLF16, MYBL2, MYC, NFYB, POU3F1, SMAD1, SOX3, SP5, TCF12, TFDP1, TP53, ZIC3, and ZNF195.

In some embodiments, the one or more additional rejuvenation-promoting genes are selected from the group consisting of CEBPZ, MYBL2, MYC, NFYB and ZNF195.

In some embodiments, the one or more additional rejuvenation-promoting genes are selected from the group consisting of ACTB, AKR1C1, ANAPC11, AP2B1, APLP2, APOD, ARF5, ARL4A, ARPC3, ARPP19, ATOX1, ATP5F1E, ATP5MC1, ATP5MC3, ATP5MD, ATP5ME, ATP5MF, ATP5MG, ATP5MPL, ATP5PF, ATP6V0B, ATP6V0E1, ATXN7L3B, B2M, B3GAT2, BEX1, BEX3, BEX5, BLOC1S1, BMERB1, C18orf32, C1orf122, C1QBP, C4orf48, CADM4, CALM1, CALM3, CALR, CANX, CAV2, CC2D1A, CCND1, CCNI, CD63, CD82, CDC42, CDH2, CFL1, CHCHD2, CHGB, CIAO2B, CLCN3, CLTA, CLTC, CNN3, CNTN1, COTL1, COX411, COX6A1, COX6C, COX7A2, COX7C, COX8A, CPNE8, CPS1, CRNDE, CSPG4, CTHRC1, CUL4B, CYP51A1, DBI, DCX, DDAH1, DDX1, DENND10, DMD, DMRT2, DNAJA2, DPYSL2, DRAP1, DSTN, DYNC112, EDF1, EDIL3, EEF1A1, EEF1B2, EEF2, EID1, EIF3J, ELOB, EMC10, EMP2, ESD, ETV1, FABP7, FAM171B, FAM177A1, FAU, FIS1, FXYD6, GADD45A, GAP43, GCSH, GNAS, GOLM1, GPM6B, GSTP1, H3-3A, H3-3B, HINT1, HNRNPA1, HNRNPA3, HNRNPAB, HNRNPC, HNRNPK, HNRNPM, HNRNPR, HSPA5, IGFBP2, ITGB8, ITM2A, ITM2B, JPT1, KDELR1, KLRK1-AS1, KRTCAP2, KTN1, LDHB, LHFPL3, LRRC4B, LY6H, MAP2, MARCKS, MARCKSL1, MIA, MICOS10, MIF, MIR9-1HG, MMGT1, MPZL1, MT3, MTLN, MTRNR2L12, MTRNR2L8, MYL12A, MYL12B, NACA, NARS1, NCL, NDUFA1, NDUFA11, NDUFA13, NDUFA3, NDUFA4, NDUFB1, NDUFB11, NDUFB2, NDUFB6, NDUFB7, NDUFC2, NDUFS5, NEU4, NUCKS1, OAZ1, OLFM2, OSBPL8, OST4, OSTC, PABPC1, PCBP2, PCDH10, PCDH11X, PCDH17, PCDHB2, PCDHGB6, PDGFRA, PDIA6, PEBP1, PEG10, PFN1, PGRMC1, PKIA, PLPP3, PLPPR1, PPIA, PRDX1, PRDX2, PRDX5, PSMB1, PSMB9, PTMS, PTN, PTPRA, RAB10, RAB14, RAB2A, RAB31, RAC1, RACK1, RMDN2, RAMP1, RO60, ROBO1, RRAGB, RTN3, S100B, SARAF, SAT1, SBDS, SCARB2, SCP2, SCRG1, SEC62, SELENOK, SELENOT, SELENOW, SERF2, SERPINE2, SET, SH3BGRL, SKP1, SLC25A6, SLIT2, SLITRK2, SMC3, SMDT1, SMOC1, SMS, SNCA, SNHG29, SNHG6, SNX3, SNX22, SOD1, SOX11, SOX2, SOX9, SPCS2, SPCS3, SRP14, SSR4, STAG2, STMN1, SUPT16H, TALDO1, TBCB, TCEAL7, TCEAL8, TCEAL9, TIMP1, TLE5, TM4SF1, TM9SF3, TMA7, TMBIM6, TMCO1, TMEM147, TMEM258, TMEM50A, TMOD2, TMSB10, TMSB4X, TPT1, TRAF4, TRIO, TSC22D4, TSPAN6, TSPAN7, TTC3, TUBB, UBA52, UBL5, UQCR10, UQCR11, UQCRB, VIM, WSB2, WSCD1, YBX1, YWHAB, YWHAE, ZFAS1, ZNF428, and ZNF462.

In some embodiments, the one or more additional rejuvenation-promoting genes are selected from the group consisting of APOD, B2M, BEX3, BEX5, CCND1, CTHRC1, EDIL3, EMC10, FABP7, GADD45A, ITM2A, LRRC4B, LY6H, MIA, MT3, NEU4, OLFM2, PTMS, RAMP1, SNX3, TRAF4, TRIO, UBA52, and YWHAB.

In some embodiments, the one or more additional rejuvenation-promoting genes are selected from the group consisting of ANAPC11, APOD, ATP5MC3, B2M, CALM1, MT3, NEU4, PEBP1, RAMP1, SOD1 and TBCB.

In some embodiments, the one or more additional rejuvenation-promoting genes are selected from the group consisting of APOD, BEX3, BEX5, CCND1, CTHRC1, EDIL3, EMC10, GADD45A, ITM2A, MIA, TRAF4, and TRIO.

In some embodiments, the one or more additional rejuvenation-promoting genes are selected from the group consisting of B2M, FABP7, LRRC4B, LY6H, MT3, NEU4, OLFM2, PTMS, RAMP1, SNX3, UBA52, and YWHAB.

In some embodiments, the one or more additional rejuvenation-promoting genes are selected from the group consisting of LY6H, MIA, GADD45A, ITM2A and ITM2B.

In some embodiments, the one or more additional rejuvenation-promoting genes are selected from the group consisting of HDAC2, EZH2, MYC, HMGA2, NFIB and TEAD2.

In some embodiments, the method further comprises the step of inhibiting expression of one or more rejuvenation-suppressing genes in the population of glial progenitor cells.

In some embodiments, the one or more rejuvenation-suppressing genes are selected from the group consisting of ABCG1, ADGRB1, ADGRG1, AKAP9, AL360181.3, ANKRD10, ARGLU1, ARL4C, ARL16, ARMCX6, ATP1A2, ATP1B3, ATP10B, B3GNT7, BHLHE41, BPTF, BRI3, BX664615.2, BX890604.1, C1QL2, CAMK2N1, CCDC85B, CCNL1, CHCHD10, CHORDC1, CIRBP, CLDN10, COL9A1, COL9A2, CXADR, DANCR, DCXR, DHX36, DLL3, DNAJA1, DNM3, ECH1, EGR1, EIF1AX, ELAVL3, EMID1, ETFB, FABP5, FAM133A, FAM133B, FBXO2, FERMT1, FIBIN, FOS, FOSB, FSCN1, FSIP2, GABPB1-AS1, GALR1, GNG8, GNPTAB, GOLGA8A, GOLGA8B, GPR155, GRID2, GRM7, HAPLN1, HMX1, HSPA1A, HSPA1B, HSPH1, HTRA1, IGFBP2, JAG1, JUN, JUNB, KCNIP4, KCNQ1OT1, KLF3-AS1, LAMP2, LINC01116, LINC01301, LINC01896, LRP4, LRRC7, MACF1, MALAT1, MAP3K13, MASP1, MDH1, MT1E, MT2A, MYT1, NASP, NKTR, NUTM2A-AS1, OFD1, PCDHB5, PCDHGA3, PCDHGB6, PEPD, PHGDH, PLCG2, PMP2, PNISR, PPP1R14A, PTGDS, RAB3IP, RAF1, RAP1GAP, RARRES2, RBM25, RBMX, REV3L, RHOBTB3, RIMS2, RIT2, RRBP1, RSRP1, S100A1, S100A16, SAT1, SCG2, SEMA3E, SERTAD1, SEZ6L, SEZ6L2, SH3GLB2, SNHG15, SNRNP70, SPARCL1, SRSF5, STAT3, STXBP6, SYNRG, THBS4, TLE4, TMEM176B, TPI1, TSC22D3, USP11, VCAN, WFDC1, WSB1, ZFYVE16, ZNF528, and ZNF528-AS1.

In some embodiments, the one or more rejuvenation-suppressing genes are selected from the group consisting of ADGRG1 ARL4C, ARMCX6, ATP1A2, ATP1B3, B3GNT7, CXADR, DLL3, FABP5, FIBIN, IGFBP2, LRRC7, MAP3K13, MT1E, MT2A, PCDHGA3, PCDHGB6, PLCG2, PTGDS, SAT1, SEZ6L, SPARCL1, THBS4, and TLE4

In some embodiments, the one or more rejuvenation-suppressing genes are selected from the group consisting of ARGLU1, EGR1, FSIP2, HSPH1, MACF1, NKTR, RBMX, STAT3, TLE4, and WSB1.

In some embodiments, the one or more rejuvenation-suppressing genes are selected from the group consisting of ADGRG1, ATP1A2, ATP1B3, B3GNT7, CXADR, DLL3, FABP5, MT1E, MT2A, PTGDS, SEZ6L, and THBS4.

In some embodiments, the one or more rejuvenation-suppressing genes are selected from the group consisting of ARL4C, ARMCX6, FIBIN, IGFBP2, LRRC7, MAP3K13, PCDHGA3, PCDHGB6, PLCG2, SAT1, SPARCL1, and TLE4.

In some embodiments, the one or more rejuvenation-suppressing genes are selected from the group consisting of ZNF274, MAX, E2F6, IKZF3 and STAT3.

In some embodiments, the step of inhibiting expression of one or more rejuvenation-suppressing genes comprises administering, to the population of glial progenitor cells, a small molecule inhibitor of a rejuvenation-suppressing gene. In some embodiments, the small molecule inhibitor is an AHR inhibitor selected from the group consisting of BAY-218, perillaldehyde StemRegenin 1 (SR1), KYN-101, CH-223191, BAY 2416964, PDM2 and GNF351.

In some embodiments, the step of inhibiting expression of one or more rejuvenation-suppressing genes comprises administering, to the population of glial progenitor cells, an expression vector encoding one or more microRNAs. In some embodiments, the one or more microRNAs are selected from the group consisting of miR-193a-5P, miR-23b-3-p, miR-4687-3p, miR-4651, miR-4270 and miR-24-3p.—Need to expand the group.

In some embodiments, the step of inhibiting expression of one or more rejuvenation-suppressing genes comprises administering, to the population of glial progenitor cells, an expression vector encoding one or more shRNAs.

In some embodiments, the step of inhibiting expression of one or more rejuvenation-suppressing genes comprises administering, to the population of glial progenitor cells, one or more antisense oligonucleotides, or an expression vector encoding one or more antisense oligonucleotides.

In some embodiments, the step of inhibiting expression of one or more rejuvenation-suppressing genes comprises administering, to the population of glial progenitor cells, an expression vector encoding a nuclease-based gene editing system. Examples of nuclease-based gene editing systems include, but are not limited to, CRISPR-CAS system, ZFN system, and TALEN system.

### III. Expression Vectors

Another aspect of the present application relates to an expression vector described in the present application.

In some embodiments, the expression vector comprises a nucleotide sequence encoding human BCL11A or a functional variant thereof. In some embodiments, the expression vector comprises a nucleotide sequence encoding human BCL11A. In some embodiments, the expression vector comprises a nucleotide sequence encoding SEQ ID NO:2.

In some embodiments, the regulatory element comprises a ubiquitous promoter, such as chicken beta-actin (CBA) promoter, hybrid form of the CBA promoter (CBh) CAG promoter, cytomegalovirus (CMV) promoter and rous sarcoma virus (RSV) promoter.

In some embodiments, the regulatory element comprises a promoter and/or enhance of a gene which is selectively expressed by glial progenitor cells, such as the promoter/enhancer of platelet derived growth factor alpha (PDGFRA), zinc finger protein 488 (ZNF488), G protein-coupled receptor (GPR17), oligodendrocyte Transcription Factor 2 (OLIG2), chondroitin sulfate proteoglycan 4 (CSPG4), and SRY-box transcription factor 10 (SOX10).

In some embodiments, the regulatory element comprises an inducible promoter. In some embodiments, the inducible promoter is a tet-on or tetp-off promoter. In some embodiments, the inducible promoter a cumate-controlled operator system, wherein transcription of the gene of interest is activated in the presence of cumate. In some embodiments, the expression vector of the present application comprises a cumate-controlled operator system, wherein transcription of the gene of interest is activated in the absence of cumate. In some embodiments, the inducible promoter comprises a rapamycin-controlled operator system, wherein transcription of the gene of interest is activated in the presence of rapamycin.

In some embodiments, the expression vector is capable of expressing an agent that inhibits the activity of an endogenous repressor of BCL11A activity in the adult glial progenitor cells. Examples of repressor of BCL11A activity include, but are not limited to, KLF1, POGZ, HRI, Mi2β, SOX2 and FOXQ1.

In some embodiments, the expression vector is capable of expressing one or more rejuvenation-promoting genes in a population of glial progenitor cells.

In some embodiments, the one or more additional rejuvenation-promoting genes are selected from the group consisting of ARX, CEBPZ, DLX1, DLX2, ELK1, ETS1, ETV4, KLF16, MYBL2, MYC, NFYB, POU3F1, SMAD1, SOX3, SP5, TCF12, TFDP1, TP53, ZIC3, and ZNF195.

In some embodiments, the one or more additional rejuvenation-promoting genes are selected from the group consisting of CEBPZ, MYBL2, MYC, NFYB and ZNF195.

In some embodiments, the one or more additional rejuvenation-promoting genes are selected from the group consisting of ACTB, AKR1C1, ANAPC11, AP2B1, APLP2, APOD, ARF5, ARL4A, ARPC3, ARPP19, ATOX1, ATP5F1E, ATP5MC1, ATP5MC3, ATP5MD, ATP5ME, ATP5MF, ATP5MG, ATP5MPL, ATP5PF, ATP6V0B, ATP6V0E1, ATXN7L3B, B2M, B3GAT2, BEX1, BEX3, BEX5, BLOC1S1, BMERB1, C18orf32, C1orf122, C1QBP, C4orf48, CADM4, CALM1, CALM3, CALR, CANX, CAV2, CC2D1A, CCND1, CCNI, CD63, CD82, CDC42, CDH2, CFL1, CHCHD2, CHGB, CIAO2B, CLCN3, CLTA, CLTC, CNN3, CNTN1, COTL1, COX411, COX6A1, COX6C, COX7A2, COX7C, COX8A, CPNE8, CPS1, CRNDE, CSPG4, CTHRC1, CUL4B, CYP51A1, DBI, DCX, DDAH1, DDX1, DENND10, DMD, DMRT2, DNAJA2, DPYSL2, DRAP1, DSTN, DYNC112, EDF1, EDIL3, EEF1A1, EEF1B2, EEF2, EID1, EIF3J, ELOB, EMC10, EMP2, ESD, ETV1, FABP7, FAM171B, FAM177A1, FAU, FIS1, FXYD6, GADD45A, GAP43, GCSH, GNAS, GOLM1, GPM6B, GSTP1, H3-3A, H3-3B, HINT1, HNRNPA1, HNRNPA3, HNRNPAB, HNRNPC, HNRNPK, HNRNPM, HNRNPR, HSPA5, IGFBP2, ITGB8, ITM2A, ITM2B, JPT1, KDELR1, KLRK1-AS1, KRTCAP2, KTN1, LDHB, LHFPL3, LRRC4B, LY6H, MAP2, MARCKS, MARCKSL1, MIA, MICOS10, MIF, MIR9-1HG, MMGT1, MPZL1, MT3, MTLN, MTRNR2L12, MTRNR2L8, MYL12A, MYL12B, NACA, NARS1, NCL, NDUFA1, NDUFA11, NDUFA13, NDUFA3, NDUFA4, NDUFB1, NDUFB11, NDUFB2, NDUFB6, NDUFB7, NDUFC2, NDUFS5, NEU4, NUCKS1, OAZ1, OLFM2, OSBPL8, OST4, OSTC, PABPC1, PCBP2, PCDH10, PCDH11X, PCDH17, PCDHB2, PCDHGB6, PDGFRA, PDIA6, PEBP1, PEG10, PFN1, PGRMC1, PKIA, PLPP3, PLPPR1, PPIA, PRDX1, PRDX2, PRDX5, PSMB1, PSMB9, PTMS, PTN, PTPRA, RAB10, RAB14, RAB2A, RAB31, RAC1, RACK1, RMDN2, RAMP1, RO60, ROBO1, RRAGB, RTN3, S100B, SARAF, SAT1, SBDS, SCARB2, SCP2, SCRG1, SEC62, SELENOK, SELENOT, SELENOW, SERF2, SERPINE2, SET, SH3BGRL, SKP1, SLC25A6, SLIT2, SLITRK2, SMC3, SMDT1, SMOC1, SMS, SNCA, SNHG29, SNHG6, SNX3, SNX22, SOD1, SOX11, SOX2, SOX9, SPCS2, SPCS3, SRP14, SSR4, STAG2, STMN1, SUPT16H, TALDO1, TBCB, TCEAL7, TCEAL8, TCEAL9, TIMP1, TLE5, TM4SF1, TM9SF3, TMA7, TMBIM6, TMCO1, TMEM147, TMEM258, TMEM50A, TMOD2, TMSB10, TMSB4X, TPT1, TRAF4, TRIO, TSC22D4, TSPAN6, TSPAN7, TTC3, TUBB, UBA52, UBL5, UQCR10, UQCR11, UQCRB, VIM, WSB2, WSCD1, YBX1, YWHAB, YWHAE, ZFAS1, ZNF428, and ZNF462.

In some embodiments, the one or more additional rejuvenation-promoting genes are selected from the group consisting of APOD, B2M, BEX3, BEX5, CCND1, CTHRC1, EDIL3, EMC10, FABP7, GADD45A, ITM2A, LRRC4B, LY6H, MIA, MT3, NEU4, OLFM2, PTMS, RAMP1, SNX3, TRAF4, TRIO, UBA52, and YWHAB.

In some embodiments, the one or more additional rejuvenation-promoting genes are selected from the group consisting of ANAPC11, APOD, ATP5MC3, B2M, CALM1, MT3, NEU4, PEBP1, RAMP1, SOD1 and TBCB.

In some embodiments, the one or more additional rejuvenation-promoting genes are selected from the group consisting of APOD, BEX3, BEX5, CCND1, CTHRC1, EDIL3, EMC10, GADD45A, ITM2A, MIA, TRAF4, and TRIO.

In some embodiments, the one or more additional rejuvenation-promoting genes are selected from the group consisting of B2M, FABP7, LRRC4B, LY6H, MT3, NEU4, OLFM2, PTMS, RAMP1, SNX3, UBA52, and YWHAB.

In some embodiments, the one or more additional rejuvenation-promoting genes are selected from the group consisting of LY6H, MIA, GADD45A, ITM2A and ITM2B.

In some embodiments, the one or more additional rejuvenation-promoting genes are selected from the group consisting of HDAC2, EZH2, MYC, HMGA2, NFIB and TEAD2.

In some embodiments, the expression vector is capable of expressing an repressor of one or more rejuvenation-suppressing genes in a population of glial progenitor cells.

In some embodiments, the one or more rejuvenation-suppressing genes are selected from the group consisting of ABCG1, ADGRB1, ADGRG1, AKAP9, AL360181.3, ANKRD10, ARGLU1, ARL4C, ARL16, ARMCX6, ATP1A2, ATP1B3, ATP10B, B3GNT7, BHLHE41, BPTF, BRI3, BX664615.2, BX890604.1, C1QL2, CAMK2N1, CCDC85B, CCNL1, CHCHD10, CHORDC1, CIRBP, CLDN10, COL9A1, COL9A2, CXADR, DANCR, DCXR, DHX36, DLL3, DNAJA1, DNM3, ECH1, EGR1, EIF1AX, ELAVL3, EMID1, ETFB, FABP5, FAM133A, FAM133B, FBXO2, FERMT1, FIBIN, FOS, FOSB, FSCN1, FSIP2, GABPB1-AS1, GALR1, GNG8, GNPTAB, GOLGA8A, GOLGA8B, GPR155, GRID2, GRM7, HAPLN1, HMX1, HSPA1A, HSPA1B, HSPH1, HTRA1, IGFBP2, JAG1, JUN, JUNB, KCNIP4, KCNQ1OT1, KLF3-AS1, LAMP2, LINC01116, LINC01301, LINC01896, LRP4, LRRC7, MACF1, MALAT1, MAP3K13, MASP1, MDH1, MT1E, MT2A, MYT1, NASP, NKTR, NUTM2A-AS1, OFD1, PCDHB5, PCDHGA3, PCDHGB6, PEPD, PHGDH, PLCG2, PMP2, PNISR, PPP1R14A, PTGDS, RAB3IP, RAF1, RAP1GAP, RARRES2, RBM25, RBMX, REV3L, RHOBTB3, RIMS2, RIT2, RRBP1, RSRP1, S100A1, S100A16, SAT1, SCG2, SEMA3E, SERTAD1, SEZ6L, SEZ6L2, SH3GLB2, SNHG15, SNRNP70, SPARCL1, SRSF5, STAT3, STXBP6, SYNRG, THBS4, TLE4, TMEM176B, TPI1, TSC22D3, USP11, VCAN, WFDC1, WSB1, ZFYVE16, ZNF528, and ZNF528-AS1.

In some embodiments, the one or more rejuvenation-suppressing genes are selected from the group consisting of ADGRG1 ARL4C, ARMCX6, ATP1A2, ATP1B3, B3GNT7, CXADR, DLL3, FABP5, FIBIN, IGFBP2, LRRC7, MAP3K13, MT1E, MT2A, PCDHGA3, PCDHGB6, PLCG2, PTGDS, SAT1, SEZ6L, SPARCL1, THBS4, and TLE4.

In some embodiments, the one or more rejuvenation-suppressing genes are selected from the group consisting of ARGLU1, EGR1, FSIP2, HSPH1, MACF1, NKTR, RBMX, STAT3, TLE4, and WSB1.

In some embodiments, the one or more rejuvenation-suppressing genes are selected from the group consisting of ADGRG1, ATP1A2, ATP1B3, B3GNT7, CXADR, DLL3, FABP5, MT1E, MT2A, PTGDS, SEZ6L, and THBS4.

In some embodiments, the one or more rejuvenation-suppressing genes are selected from the group consisting of ARL4C, ARMCX6, FIBIN, IGFBP2, LRRC7, MAP3K13, PCDHGA3, PCDHGB6, PLCG2, SAT1, SPARCL1, and TLE4.

In some embodiments, the one or more rejuvenation-suppressing genes are selected from the group consisting of ZNF274, MAX, E2F6, IKZF3 and STAT3.

In some embodiments, the repressor comprises one or more microRNAs. In some embodiments, the one or more microRNAs are selected from the group consisting of miR-193a-5P, miR-23b-3-p, miR-4687-3p, miR-4651, miR-4270 and miR-24-3p.—Need to expand the group.

In some embodiments, the repressor comprises one or more shRNAs.

In some embodiments, the repressor comprises one or more antisense oligonucleotides.

In some embodiments, the repressor comprises a nuclease-based gene editing system. Examples of nuclease-based gene editing systems include, but are not limited to, CRISPR-CAS system, ZFN system, and TALEN system.

In some embodiments, the expression vector of the present application is a non-viral vector. In some embodiments, the non-viral vector is a plasmid. In some embodiment, the non-viral vector is a bacterial vector.

In some embodiments, the expression vector of the present application is a viral vector. In some embodiments, the viral vector is a lentiviral vector, an AAV vector or a retroviral vector.

In some embodiments, the expression vector of the present application is a lentiviral vector.

In some embodiments, the expression vector of the present application is an AAV vector.

Another aspect of the present application relates a cell harboring an expression vector of the present application. As used herein, the phrase “cell harboring an expression vector” refers to a cell that comprises an expression vector either in the integrated form or non-integrated (epichromosomal) form.

A further aspect of the present disclosure is directed to a preparation of glial progenitor cells expressing the genetic construct according to the present disclosure.

The examples below are intended to exemplify the practice of embodiments of the disclosure but are by no means intended to limit the scope thereof.

### EXAMPLES

### Example 1: Materials and Methods

Generation and Aging of hGPCs

Human embryonic stem cells (hESCs; line WA09/H9) and induced pluripotent stem cells (iPSCs; line C27) were expanded in feeder-free conditions in mTeSR1 medium, then differentiated into hGPCs. To assess the effects of age on hGPCs in vitro, hGPCs maintained on mouse laminin were raised to 160 DIV, and passaged by manual dissection every three weeks or at 90% confluency for 360 DIV. Cells were harvested for analysis at DIV 0, 30, 60, 90, 180, and 360.

Lentivirus Production and Infection In Vitro

The study identified BCL11A isoform 2 as the most represented in isolated hESC-derived GPCs. On that basis, BCL11A isoform 2 (NCBI Reference Sequence: NM_018014.4) was cloned into the pTANK backbone followed by T2A-EGFP, driven by the ubiquitously expressed CBh promoter. To generate an EGFP-expressing control virus, EGFP alone was cloned downstream of the CBh promoter. Viral particles were produced by transfection of 293HEK cells, concentrated and titrated (LV-BCL11A: 3.83×10\hat{ }9 PFU/mL, LV-EGFP: 4.50×10\hat{ }9 PFU/mL). GPCs were transduced in vitro by adding either LV-BCL11A or LV-EGFP to the culture media for 24 hours at an MOI of 2. One week after infection, cells were fixed with 4% paraformaldehyde (PFA) or dissociated for downstream analysis and processing.

Etoposide Treatment in hGPCs

At 160 DIV, hGPCs were treated with either 5 μM etoposide or dimethyl sulfoxide (DMSO) as a control, added to standard glial culture medium. After 5 days of treatment, etoposide and DMSO were removed via a fresh media exchange, and cells in both conditions were treated with LV-BCL11A or LV-EGFP. A week after infection, cells were dissociated and processed for RT-QPCR as described herein.

Generation and Analysis of Glial-Chimerized Mice

For the establishment of human glial chimeric mice, hGPCs were derived from iPSCs (line C27) tagged with a cassette expressing membrane-bound tRFP and intracellular mScarlet integrated in the AAVS1 locus, enabling fluorescence-associated cell sorting and visualization of human cells via histology. At postnatal day 1, Rag1 immunodeficient mice were engrafted in the corpus callosum (CC) with 300k cells. Chimerized mice were allowed to age for 2 years following engraftment, after which they received stereotactic injection of LV-BCL11A in the left hemisphere and LV-EGFP control virus in the right hemisphere. Virus was deposited in the striatum, CC, and cortex, 1 uL per stop. Three weeks after injection, mice were euthanized and microdissected for cell sorting, or they were perfused with 4% PFA for cryosectioning and immunohistochemistry.

Immunostaining and Quantification

In vitro After fixation, cell cultures were washed with PBS and incubated in staining buffer (0.3% Triton X-100 and 1% BSA in PBS). Primary antibodies against BCL11A, GFP, or MKI67 were added for 1 hour, then cells were once again washed with PBS. Secondary antibodies and DAPI were added for 30 minutes, then cells were washed and stored in PBS containing thimerosal. Immunostained cultures were imaged on the ImageXpress High-Content Imaging System and quantified using MetaXpress software.

In vivo Cryosections were mounted and stained for GFP, human nuclear antigen, MKI67, BCL11A, PDGFRA, or OLIG2 by incubation at 4° C. overnight in permeabilization/blocking buffer (0.1% Triton X-100 and 10% normal goat serum in PBS). After rinsing with PBS, sections were incubated with secondary antibodies, rinsed, and mounted for imaging. Images were processed using ImageJ. For each mouse, MKI67+ or BCL11A+ cells were counted in the CC in 2 sections around the site of injection, and the area of the CC measured.

Cell Sorting

From culture For analysis of RNA and chromatin in bulk or single-cell, cells were isolated via fluorescence-associated cell sorting. In brief, hGPC cultures were dissociated into single cells with 50% Accutase in DPBS, then filtered and stained with DAPI to distinguish cells that were no longer intact. EGFP+/DAPI− cells were either collected in a minimal volume of PBS for single-cell RNA-seq, lysis buffer for bulk RNA extraction, or nuclei isolation buffer for CUT&Tag (Cleavage Under Targets and Tagmentation).

From glial chimeric mice Cells were isolated from chimerized mice following perfusion with cold HBSS by microdissection of the injection tract and dissociation in papain for 45 minutes at 37° C., with gentle trituration halfway through the incubation time. Papain was quenched with ovomucoid, then the dissociated tissue was filtered and sorted for RFP+/DAPI− or GFP+/DAPI− LV-infected human cells. Viability was checked via trypan blue on a hemocytometer before proceeding to single-cell RNA sequencing.

Quantitative Real-Time PCR and RNA Sequencing

RNA was extracted from either untreated hGPC cultures or GFP+ cells using the Qiagen RNeasy Micro kit. For Q-RT PCR, RNA was reverse-transcribed, then mixed with SYBR green master mix and primers targeting BCL11A, MKI67, CDKN1A, CDKN2A, IL1A, or IL8. For RNA-seq, total RNA was prepared via the Illumina TruSeq kit.

Single-Cell RNA-Sequencing & ATAC-Seq

Sorted GFP+ cells from GPC cultures or dissociated tissue were counted, then processed using the 10× Genomics Chromium v3.1 Single Cell 3′ workflow according to manufacturer instructions. Single-cell ATAC sequencing was completed using the 10× Genomics Chromium 3′ v1.1 Single Cell ATAC kit.

CUT&Tag

To further characterize the effect of BCL11A overexpression on chromatin state, the study conducted CUT&Tag on LV-transduced hGPCs following the EpiCypher CUTANA CUT&Tag protocol, using antibodies targeting H3K4me3, H3K27me3, and H3K9me3. Nuclei were isolated, adsorbed to Concalvalin A magnetic beads, and the chromatin tagmented with PAG-Tn5.

Bioinformatic Analysis

Bulk RNA-seq data was aligned using STAR and imported into R for analysis using DESeq2. Network analysis was conducted using Qiagen Ingenuity Pathway Analysis. Single-cell RNA-seq and ATAC-seq data was processed via a custom STAR-based aligner to distinguish human from mouse cells, then analyzed using Seurat and Signac in R. Fetal & adult gene signature enrichment scores were calculated using AUCell. CUT&Tag data was aligned with bowtie2 and processed with samtools and bedtools. Peaks were called using SEACR and annotated with ChIPseeker. Differential peak enrichment was modeled using DESeq2, and tracks were visualized with Interactive Genome Viewer (IGV).

### Example 2: BCL11A Expression Corresponds to hGPC Proliferation

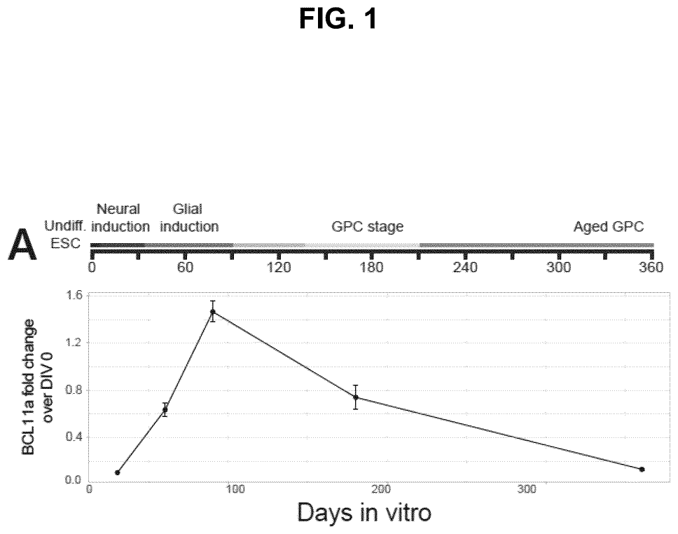

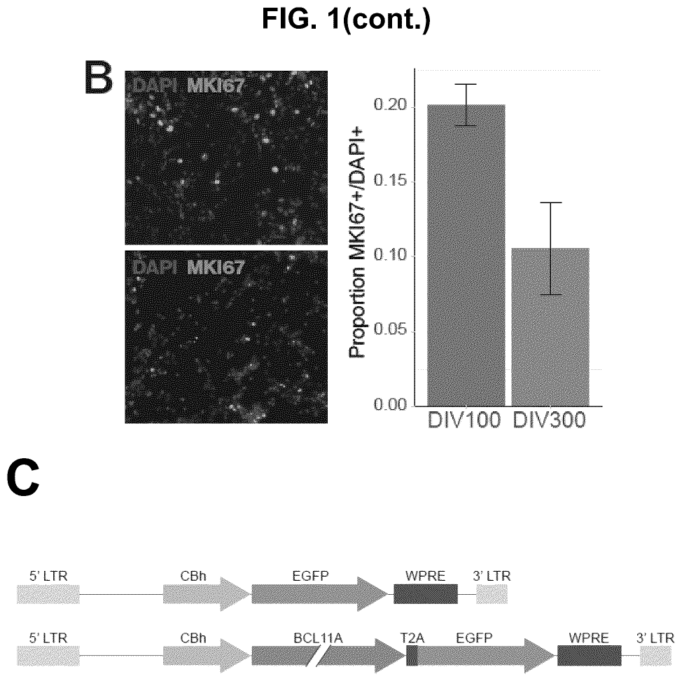

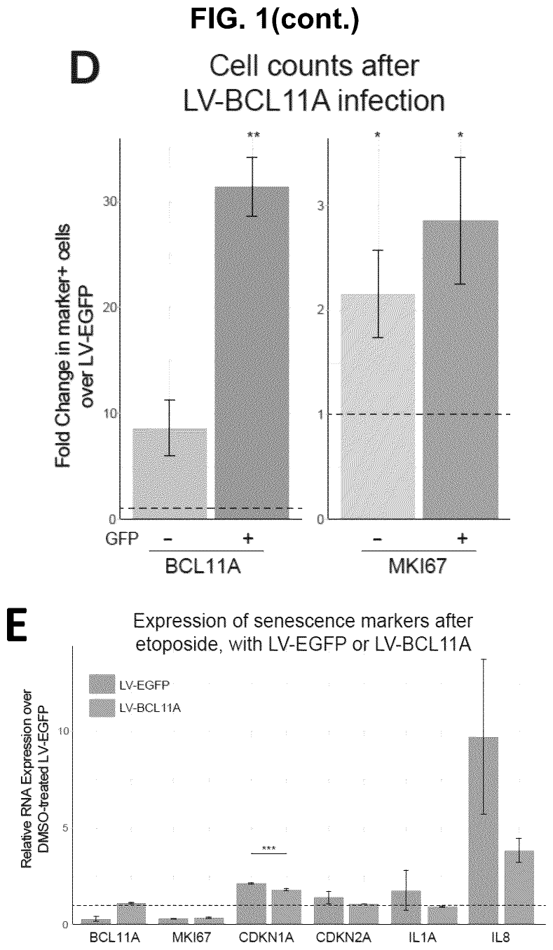

The study first asked if BCL11A is linked to hGPC mitotic competence and self-renewal. To that end, the study assessed BCL11A expression in hGPCs via RT-qPCR, during both their differentiation and subsequent in vitro aging. BCL11A expression was low during neural induction and rose during glial specification, peaking in young hGPCs at 100 DIV and declining to much lower levels by 360 DIV (FIG. 1, Panel A). The change in BCL11A expression occurred concomitantly with a reduction in cell cyclicity from 100 to 300 DIV, as assessed by MKI67 ICC (FIG. 1, Panel B).

The study next generated a lentivirus co-expressing BCL11A and EGFP, so as to identify infected cells (LV-BCL11A), along with an EGFP-only control virus (LV-EGFP) (FIG. 1, Panel C). One week after infection, hGPCs transduced with LV-BCL11A showed a significant increase in MKI67+/GFP+ cells relative to LV-EGFP controls (fold change=2.86±0.61, paired t-test *P=0.015, N=3); this was accompanied by a significant increase in cell number in the BCL11A/GFP-treated cultures relative to their LV-EGFP-treated controls (fold change over LV-EGFP=31.4±2.8, ** P=0.008 by t-test, N=3). Interestingly, non-transduced GFP− cells in those cultures that received LV-BCL11A also showed a significant increase in MKI67+ cells (log 2 fold change over LV-EGFP=2.15±0.42, paired t-test *P=0.017) suggesting that BCL11A overexpression may promote proliferation by paracrine as well as cell-intrinsic mechanisms (FIG. 1, Panel D).

To further investigate the link between self-renewal and BCL11A expression, the study treated hGPCs with etoposide, a DNA-damaging agent that halts cell division and induces marks of senescence. Following treatment with etoposide, the study observed reduced expression of BCL11A relative to DMSO-treated controls (fold change=0.30±0.14). Alongside a reduction in BCL11A expression, MKI67 fell significantly (fold change=0.35±0.04, *P=0.04 by one sample t-test, N=2) and p21 increased (fold change=2.14±0.05, *P=0.026, t-test), while other markers of senescence including p16, ILIA, and IL8 trended upwards. Treatment with LV-BCL11A restored BCL11A expression to control levels, but was not sufficient to restore MKI67 expression; however, it did result in a significant decrease in p21 expression relative to LV-EGFP (paired t-test, *** P<0.001) alongside downward trends in all p16, ILIA, and IL8, suggesting a protective effect against etoposide-induced senescence (FIG. 1, Panel E).

### Example 3: BCL11A Overexpression Induces Youth-Associated Gene Expression Signatures

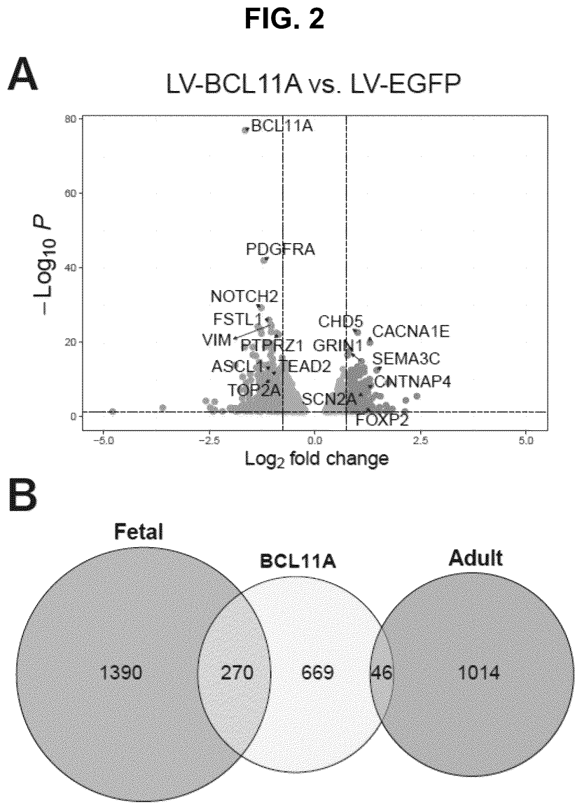

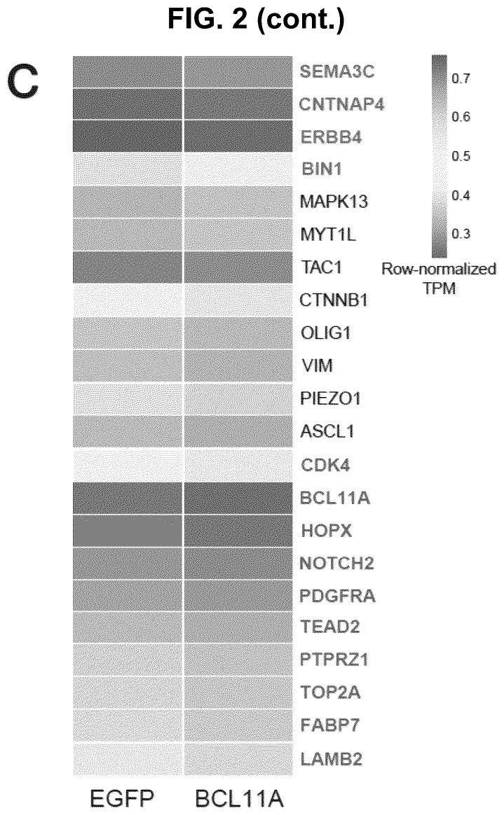

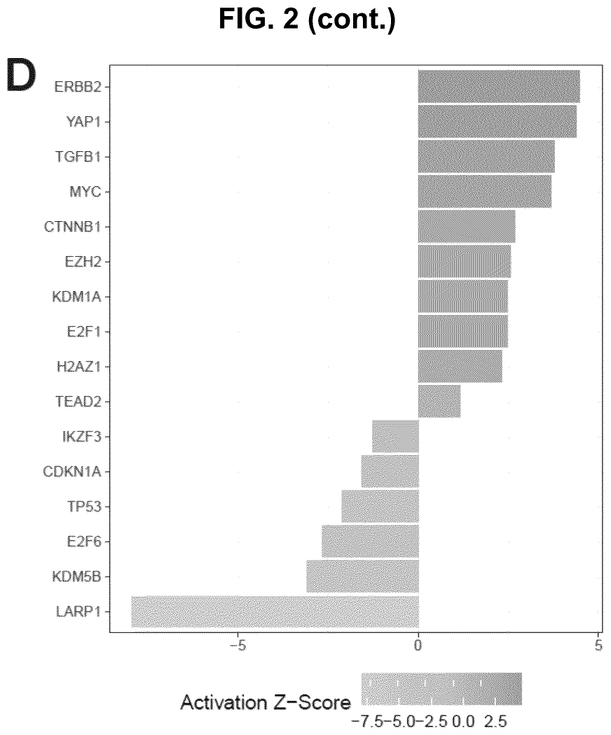

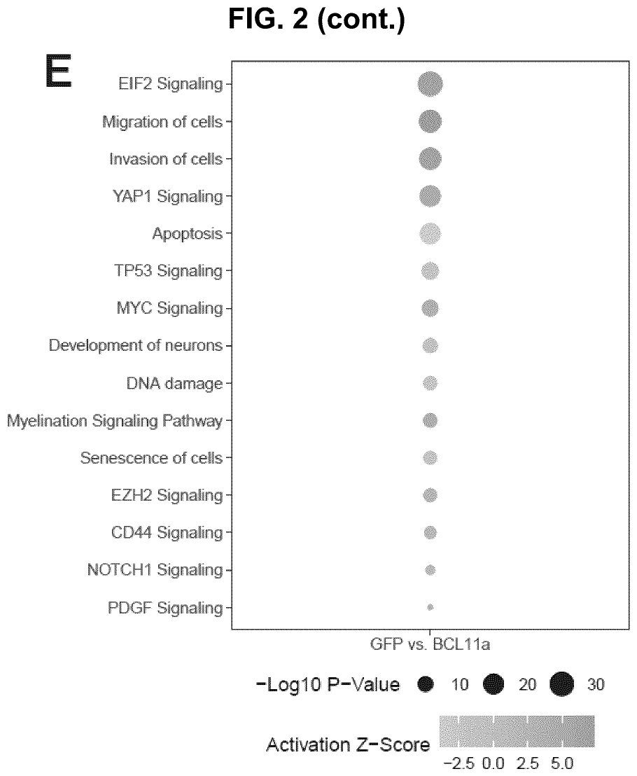

RNA sequencing from isolated LV-BCL11A or LV-EGFP-treated hGPCs revealed 985 differentially expressed genes in BCL11A-overexpressing samples, and 690 genes lower in BCL11A-infected hGPCs relative to GFP-treated controls. Upregulated genes included markers of GPC identity like PDGFRA and ASCL1, regulators of cell-cell communication like NOTCH2, and genes with roles in proliferation and migration including TOP2A and TEAD2 (FIG. 2, Panels A and C). In contrast, genes higher in control cultures included markers of mature cell identities such as MYT1L and BIN1 (FIG. 2, Panels A and C). The study then asked whether the genes upregulated following BCL11A overexpression comprised a youth-associated expression signature. The study took advantage of a generated dataset comparing the transcriptomes of hGPCs isolated from primary fetal or adult tissue, and assessed the overlap between genes induced by BCL11A and those differentially expressed in fetal samples. Of 985 genes upregulated in BCL11A-overexpressing hGPCs, 270 (27.4%) were also upregulated in fetal samples, as opposed to 46 (4.7%) in adult samples (FIG. 2, Panel B).

In order to define networks and upstream effectors responsible for inducing the youth-like transcriptional state, the study employed Ingenuity Pathway Analysis. Regulators predicted to have significant upstream activity in BCL11A-overexpressing cells included YAP1, MYC, EZH2, CTNNB1, and TEAD2. CDKN1A and TP53, markers of senescence and apoptosis, along with E2F6 and IKZF3-genes the study identified as drivers of the adult hGPC phenotype-were among those identified as repressed following BCL11A activation (FIG. 2, Panel D). The most highly activated pathways were those related to migration, invasion, and oligodendroglial identity, while pathways associated with apoptosis, neuronal identity, and senescence were repressed (FIG. 2, Panel E).

### Example 4: Single-Cell RNA Sequencing Reveals a Shift Toward Younger Cell Identity Following BCL11A Activation

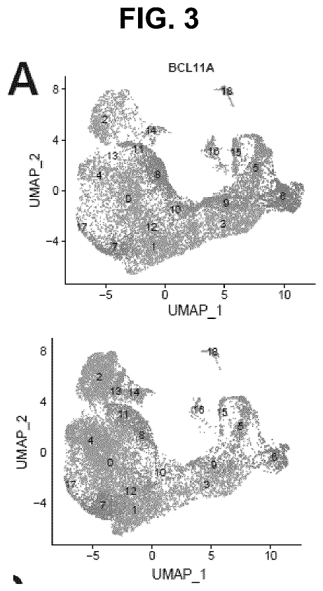

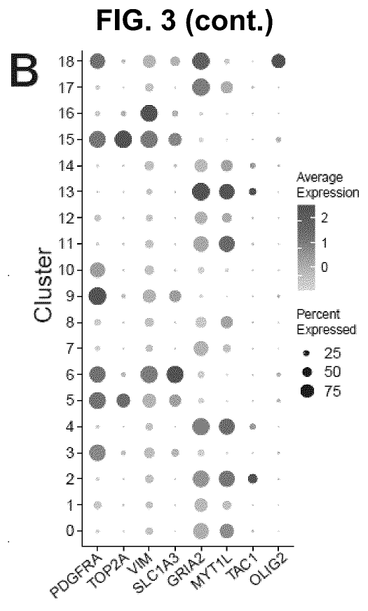

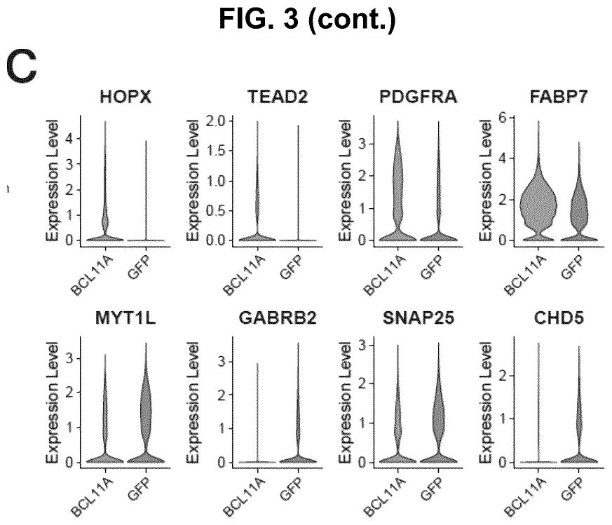

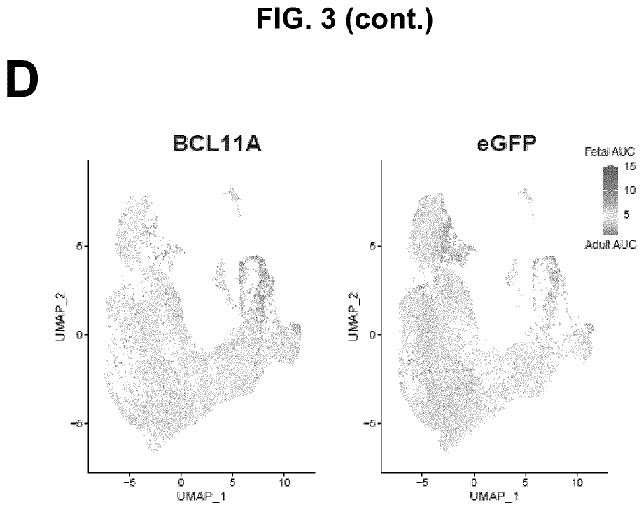

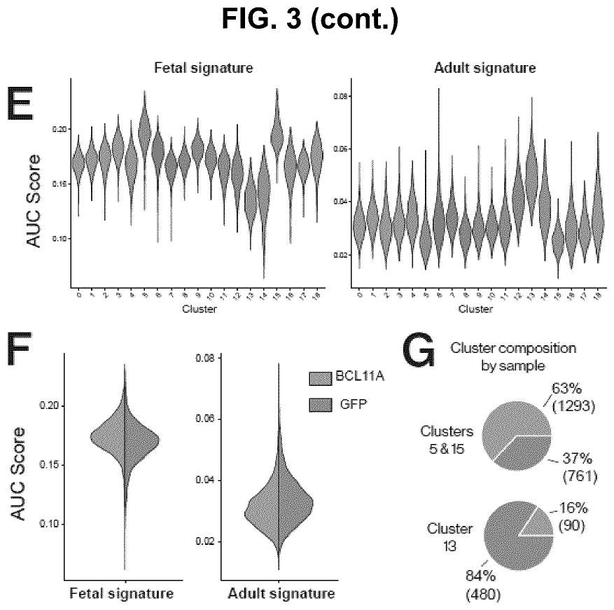

Using single-cell RNA sequencing, the study then examined the effects of BCL11A overexpression on hGPC identity in vitro. After filtering out low-quality cells, the study recovered 11,512 LV-BCL11A and 12,700 LV-GFP cells in 19 clusters (FIG. 3, Panel A). Cultures included multiple GPC subtypes: PDGFRA/ASCL1-expressing GPCs in clusters 3, 9, and 10, proliferating GPCs in clusters 5 and 15, radial glia-like GPCs marked with VIM and HOPX in cluster 16, pro-astrocytic GPCs in cluster 6, radial glia-like cells in cluster 16, pro-neural GPCs in clusters 0, 1, 4, 7, 8, 10, 11, 12, and 17, TAC1-positive neural precursors in clusters 2, 13, and 14, and OLIG2-high cells in cluster 18 (FIG. 3, Panel B). Markers of glial and, specifically, radial-glial identity were among the most enriched genes in BCL11A-treated cells, with a relative reduction in genes associated with neuronal fate (FIG. 3, Panel C). The study then compared overexpression of BCL11A to fetal and adult GPC identity by generating fetal and adult enrichment scores with AUCell, based on differentially expressed genes between fetal and adult hGPCs (FIG. 3, Panel D). Clusters 5 and 15 were the most enriched for the fetal signature, and cluster 13 the most adult-enriched (FIG. 3, Panel E); overall, BCL11A overexpression appeared to induce a shift toward fetal and away from adult identity (FIG. 3, Panel F). LV-BCL11A treated cells made up the majority of the most fetal-enriched clusters (63%), and the minority of the most adult-enriched cluster (16%) (FIG. 3, Panel G).

### Example 5: In Vivo Mobilization of Aged hGPCs in Glial-Chimeric Mice

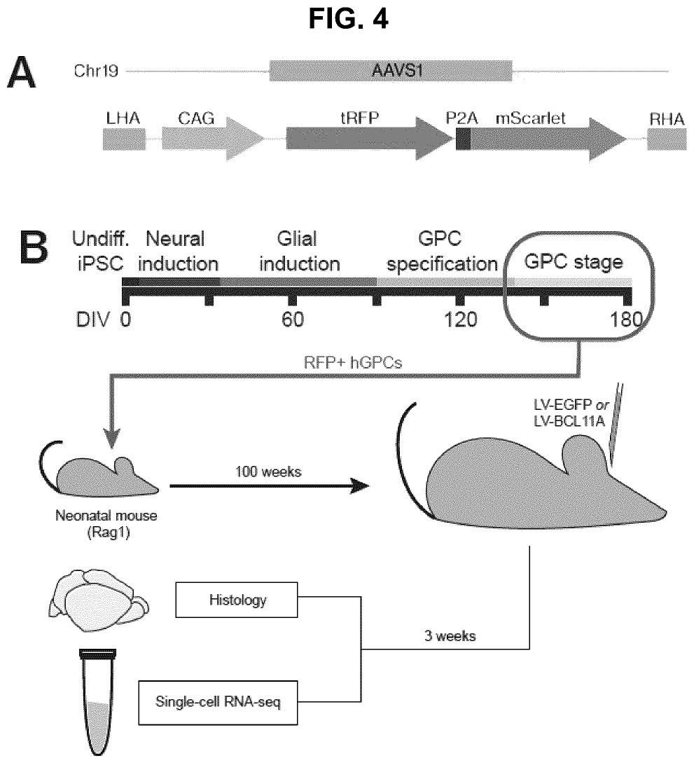

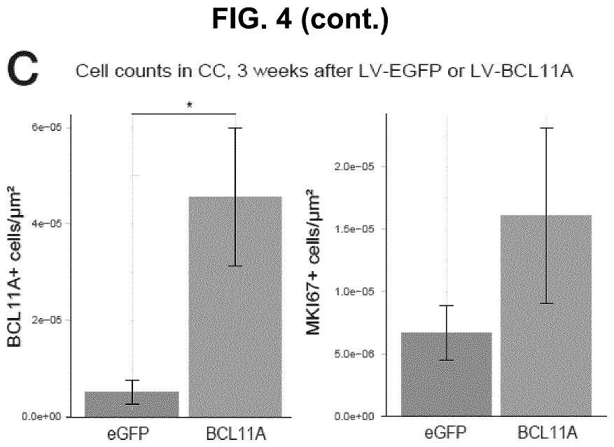

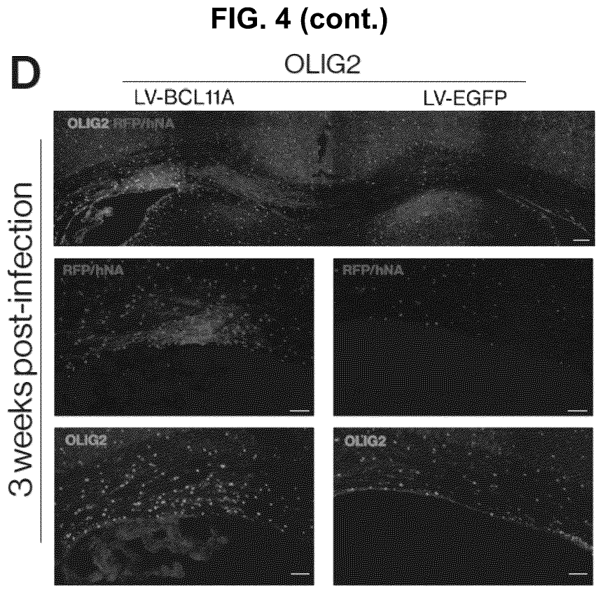

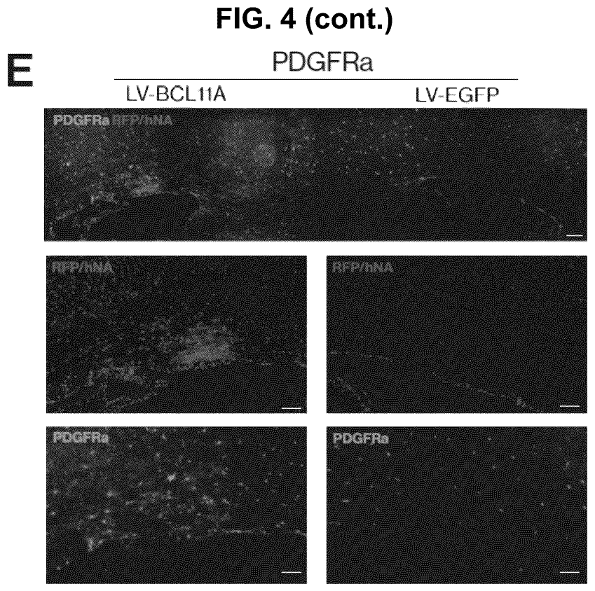

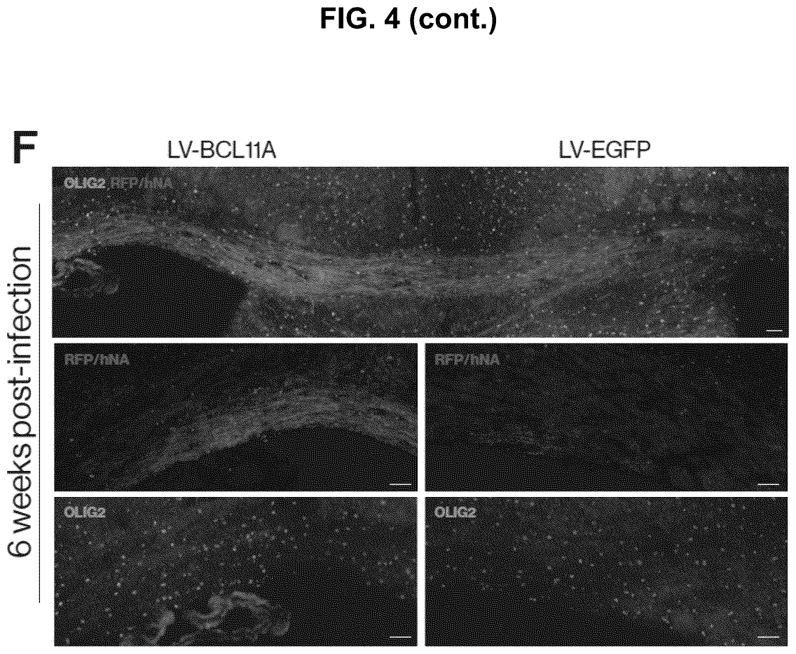

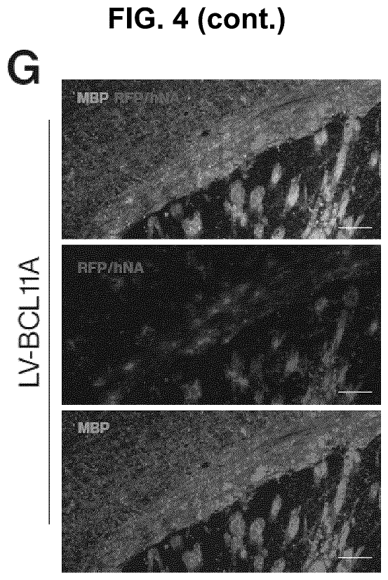

On the basis of these data, the study next asked whether BCL11A could re-initiate or accelerate self-renewal in aged GPCs. To this end, mice were engrafted with RFP+, iPSC-derived hGPCs at postnatal day 1, then allowed to age for 2 years (FIG. 4, Panel A). The study then treated these 2-year-old chimeric mice with LV-BCL11A or LV-EGFP by injection into the CC. After 3 weeks, mice were collected for single-cell transcriptomics or histological analysis (FIG. 4, Panel B). Immunostaining confirmed a significant overexpression of BCL11A in LV-BCL11A-treated hemispheres relative to their LV-EGFP-treated controls (LV-BCL11A cells/μm2=4.56e-5±8.23e-6, LV-EGFP cells/μm2=5.06e-6±1.45e-6; * P=0.031 by paired t-test, N=3). This was associated with a trend toward more MKI67+ cells in the CC of BCL11A-injected hemispheres (LV-BCL11A cells/μm2=1.6e-5±4.06e-6, LV-EGFP cells/μm2=6.63e-6±1.26e-6; P=0.079 by paired t-test, N=3) (FIG. 4, Panel C). Donor cell dispersal was both broad and relatively uniform; no tumors or heterotopias were found. The study also observed a relative increase in RFP, OLIG2+, and PDGFRa+ cells in LV-BCL11A-treated hemispheres, suggesting that BCL11A transduction activated aged GPCs to re-initiate mitotic expansion and migratory colonization of the host brain. Furthermore, the membrane-tagging of the human donor cells allowed the morphologies of a large fraction of those in the white matter to be defined as myelinating oligodendrocytes, suggesting at least partial reversal of the typical age-associated loss of myelin in these aged mice (FIG. 4, Panels D and E). Human cells colocalized with MBP in the LV-BCL11A-treated CC and striatum, indicating that the mobilized cells are able to produce myelin. This effect was still present at 6 weeks post-infection (FIG. 4, Panels F and G), with more RFP+ and OLIG2+ cells detected in the LV-BCL11A-infected hemisphere than in the LV-EGFP-treated control hemisphere.

### Example 6: BCL11A Upregulates Genes Associated with Migration and Proliferation In Vivo

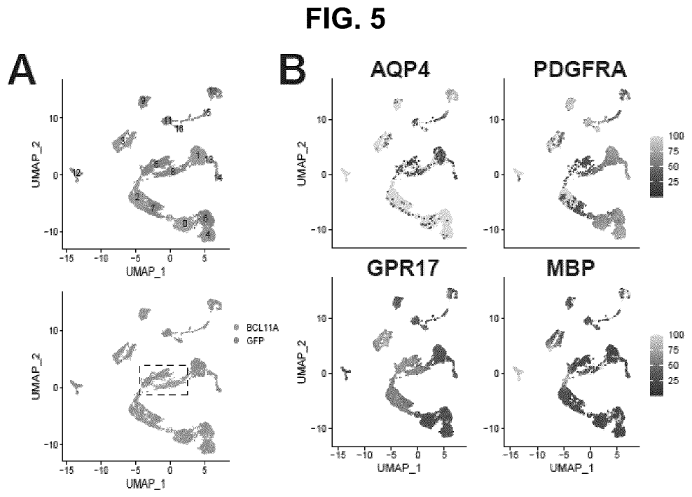

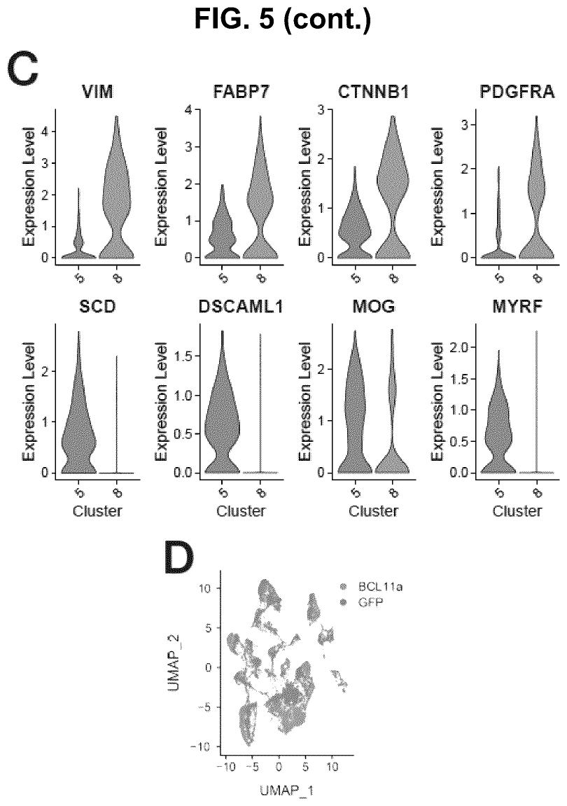

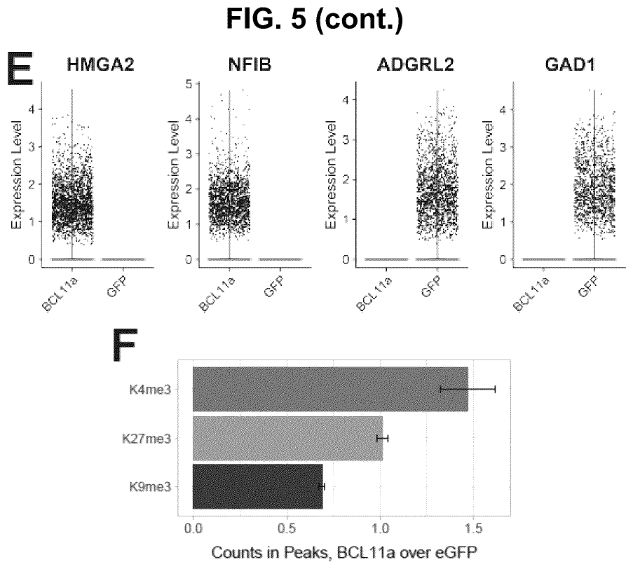

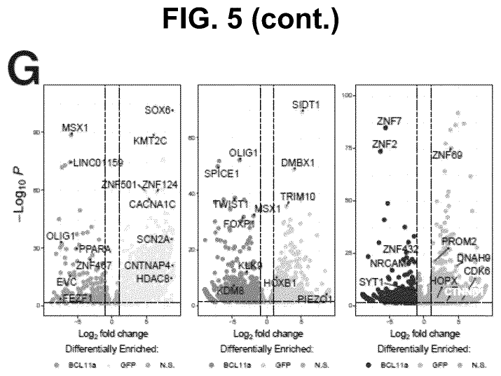

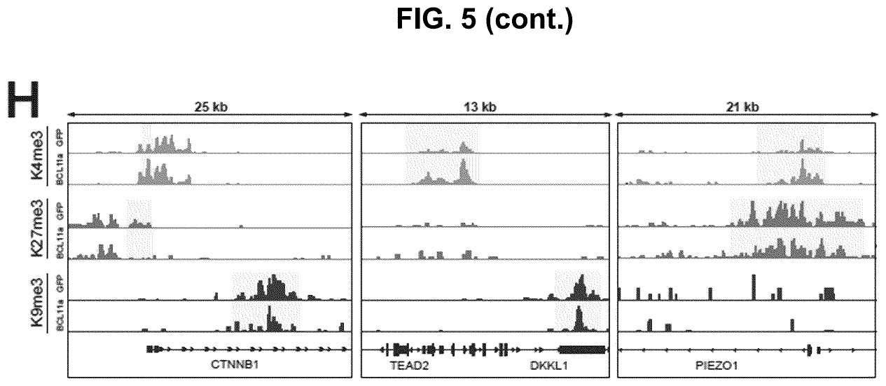

After isolating RFP+/GFP+ cells from 2-year-old chimerized mice treated with LV-BCL11A or LV-EGFP, the study conducted scRNA-seq to identify BCL11A-associated changes in vivo gene expression and phenotype. In total, the study captured 2,260 human cells from BCL11A-treated hemispheres and 2,696 from GFP-treated controls (FIG. 5, Panel A). These included AQP4+ astrocytes, PDGFRA+ GPCs, later stage GPR17+ GPCs maturing along the oligodendrocyte lineage, and a cluster of more mature MBP-defined oligodendrocytes (FIG. 5, Panel B). In particular, the study noticed a cluster of early-maturing GPCs differentially present in the BCL11A condition (FIG. 5, Panel A, bottom), and investigated this cluster further, comparing it to the most similar group of cells from the GFP control condition. In comparison to its GFP-treated counterpart, the BCL11A-treated cluster was enriched for glial progenitor genes (PDGFRA, ASCL1) and genes involved in maintenance of stemness, migration, and cell-cell signaling, including HES5 and CTNNB1. The most significantly upregulated genes were markers of astrocytes and radial glia (FABP7, VIM, HOPX), suggestive of a less-differentiated hGPC identity; in contrast, the control cells were enriched for markers of more mature glial (MOG, MYRF) and occasional neuronal (DSCAML1) differentiation (FIG. 5, Panel C).

### Example 7: BCL11A Expression Gives Rise to a Permissive Chromatin State at Youth-Associated Genes

The study next sought to investigate how BCL11A expression may modulate the chromatin state to induce a migratory, proliferative state in hGPCs. The study conducted scATAC-seq, recovering 15,801 cells from LV-GFP infected cultures and 11,465 from those transduced with LV-BCL11A (FIG. 5, Panel D). Following BCL11A overexpression, the study observed increased accessibility at loci including HMGA2 and NFIB, two targets identified as drivers of fetal hGPC identity. GFP cultures showed increased accessibility at genes associated with pro-neural pathways, including ADGRL2 and GAD1 (FIG. 5, Panel E). To understand the effects of BCL11A activation genome-wide, the study also assayed the activating chromatin mark H3K4me3, as well as the repressive marks K27me3 and K9me3. BCL11A-treated cells showed a relative enrichment in K4me3 and a relative reduction in K9me3, suggesting a genome-wide net potentiation of transcription (FIG. 5, Panel F). Of interest, OLIG1—a critical determinant of glial lineage progression—was significantly enriched for both K4me3 and K27me3 after BCL11A overexpression, indicating possible bivalent regulation of its expression (FIG. 5, Panel G). Genes identified as differentially upregulated following BCL11A overexpression also showed remodeling in their chromatin state, with a loss of repressive marks around CTNNB1, TEAD2, and PIEZO1, with concurrent gains in K4me3 signal.

Taken together, these data show that BCL11A overexpression results in a permissive chromatin state around essential genes for cell-cell communication, migration, and maintenance of stemness, including drivers of Wnt/β-catenin and YAP/TAZ signaling. Given the transcriptional upregulation of components and downstream targets of these pathways in vivo and in vitro, their activation underlies the observed mobilization of hGPCs following BCL11A expression, shifting cells toward a fetal-like state.

LIST OF SEQUENCES

| SEQ | | |
|---|---|---|
| ID | | |
| NO: | Description | Sequence |
| 1 | Human | ATGTCTCGCCGCAAGCAAGGCAAACCCCAG |
| | BCL11A | CACTTAAGCAAACGGGAATTCTCGCCCGAG |
| | (Variant 2) | CCTCTTGAAGCCATTCTTACAGATGATGAA |
| | DNA | CCAGACCACGGCCCGTTGGGAGCTCCAGAA |
| | sequence | GGGGATCATGACCTCCTCACCTGTGGGCAG |
| | | TGCCAGATGAACTTCCCATTGGGGGACATT |
| | | CTTATTTTTATCGAGCACAAACGGAAACAA |
| | | TGCAATGGCAGCCTCTGCTTAGAAAAAGCT |
| | | GTGGATAAGCCACCTTCCCCTTCACCAATC |
| | | GAGATGAAAAAAGCATCCAATCCCGTGGAG |
| | | GTTGGCATCCAGGTCACGCCAGAGGATGAC |
| | | GATTGTTTATCAACGTCATCTAGAGGAATT |
| | | TGCCCCAAACAGGAACACATAGCAGATAAA |
| | | CTTCTGCACTGGAGGGGCCTCTCCTCCCCT |
| | | CGTTCTGCACATGGAGCTCTAATCCCCACG |
| | | CCTGGGATGAGTGCAGAATATGCCCCGCAG |
| | | GGTATTTGTAAAGATGAGCCCAGCAGCTAC |
| | | ACATGTACAACTTGCAAACAGCCATTCACC |
| | | AGTGCATGGTTTCTCTTGCAACACGCACAG |
| | | AACACTCATGGATTAAGAATCTACTTAGAA |
| | | AGCGAACACGGAAGTCCCCTGACCCCGCGG |
| | | GTTGGTATCCCTTCAGGACTAGGTGCAGAA |
| | | TGTCCTTCCCAGCCACCTCTCCATGGGATT |
| | | CATATTGCAGACAATAACCCCTTTAACCTG |
| | | CTAAGAATACCAGGATCAGTATCGAGAGAG |
| | | GCTTCCGGCCTGGCAGAAGGGCGCTTTCCA |
| | | CCCACTCCCCCCCTGTTTAGTCCACCACCG |
| | | AGACATCACTTGGACCCCCACCGCATAGAG |
| | | CGCCTGGGGGCGGAAGAGATGGCCCTGGCC |
| | | ACCCATCACCCGAGTGCCTTTGACAGGGTG |
| | | CTGCGGTTGAATCCAATGGCTATGGAGCCT |
| | | CCCGCCATGGATTTCTCTAGGAGACTTAGA |
| | | GAGCTGGCAGGGAACACGTCTAGCCCACCG |
| | | CTGTCCCCAGGCCGGCCCAGCCCTATGCAA |
| | | AGGTTACTGCAACCATTCCAGCCAGGTAGC |
| | | AAGCCGCCCTTCCTGGCGACGCCCCCCCTC |
| | | CCTCCTCTGCAATCCGCCCCTCCTCCCTCC |
| | | CAGCCCCCGGTCAAGTCCAAGTCATGCGAG |
| | | TTCTGCGGCAAGACGTTCAAATTTCAGAGC |
| | | AACCTGGTGGTGCACCGGCGCAGCCACACG |
| | | GGCGAGAAGCCCTACAAGTGCAACCTGTGC |
| | | GACCACGCGTGCACCCAGGCCAGCAAGCTG |
| | | AAGCGCCACATGAAGACGCACATGCACAAA |
| | | TCGTCCCCCATGACGGTCAAGTCCGACGAC |
| | | GGTCTCTCCACCGCCAGCTCCCCGGAACCC |
| | | GGCACCAGCGACTTGGTGGGCAGCGCCAGC |
| | | AGCGCGCTCAAGTCCGTGGTGGCCAAGTTC |
| | | AAGAGCGAGAACGACCCCAACCTGATCCCG |
| | | GAGAACGGGGACGAGGAGGAAGAGGAGGAC |
| | | GACGAGGAAGAGGAAGAAGAGGAGGAAGAG |
| | | GAGGAGGAGGAGCTGACGGAGAGCGAGAGG |
| | | GTGGACTACGGCTTCGGGCTGAGCCTGGAG |
| | | GCGGCGCGCCACCACGAGAACAGCTCGCGG |
| | | GGCGCGGTCGTGGGCGTGGGCGACGAGAGC |
| | | CGCGCCCTGCCCGACGTCATGCAGGGCATG |
| | | GTGCTCAGCTCCATGCAGCACTTCAGCGAG |
| | | GCCTTCCACCAGGTCCTGGGCGAGAAGCAT |
| | | AAGCGCGGCCACCTGGCCGAGGCCGAGGGC |
| | | CACAGGGACACTTGCGACGAAGACTCGGTG |
| | | GCCGGCGAGTCGGACCGCATAGACGATGGC |
| | | ACTGTTAATGGCCGCGGCTGCTCCCCGGGC |
| | | GAGTCGGCCTCGGGGGGCCTGTCCAAAAAG |
| | | CTGCTGCTGGGCAGCCCCAGCTCGCTGAGC |
| | | CCCTTCTCTAAGCGCATCAAGCTCGAGAAG |
| | | GAGTTCGACCTGCCCCCGGCCGCGATGCCC |
| | | AACACGGAGAACGTGTACTCGCAGTGGCTC |
| | | GCCGGCTACGCGGCCTCCAGGCAGCTCAAA |
| | | GATCCCTTCCTTAGCTTCGGAGACTCCAGA |
| | | CAATCGCCTTTTGCCTCCTCGTCGGAGCAC |
| | | TCCTCGGAGAACGGGAGCTTGCGCTTCTCC |
| | | ACACCGCCCGGGGAGCTGGACGGAGGGATC |
| | | TCGGGGCGCAGCGGCACGGGAAGTGGAGGG |
| | | AGCACGCCCCATATTAGTGGTCCGGGCCCG |
| | | GGCAGGCCCAGCTCAAAAGAGGGCAGACGC |
| | | AGCGACACTTGTTCTTCACACACCCCCATT |
| | | CGGCGTAGTACCCAGAGAGCTCAAGATGTG |
| | | TGGCAGTTTTCGGATGGAAGCTCGAGAGCC |
| | | CTTAAGTTC |
| 2 | Human | MSRRKQGKPQHLSKREFSPEPLEAILTDDE |
| | BCL11A | PDHGPLGAPEGDHDLLTCGQCQMNFPLGDI |
| | (Variant 2) | LIFIEHKRKQCNGSLCLEKAVDKPPSPSPI |
| | amino acid | EMKKASNPVEVGIQVTPEDDDCLSTSSRGI |
| | sequence | CPKQEHIADKLLHWRGLSSPRSAHGALIPT |
| | | PGMSAEYAPQGICKDEPSSYTCTTCKQPFT |
| | | SAWFLLQHAQNTHGLRIYLESEHGSPLTPR |
| | | VGIPSGLGAECPSQPPLHGIHIADNNPFNL |
| | | LRIPGSVSREASGLAEGRFPPTPPLFSPPP |
| | | RHHLDPHRIERLGAEEMALATHHPSAFDRV |
| | | LRLNPMAMEPPAMDFSRRLRELAGNTSSPP |
| | | LSPGRPSPMQRLLQPFQPGSKPPFLATPPL |
| | | PPLQSAPPPSQPPVKSKSCEFCGKTFKFQS |
| | | NLVVHRRSHTGEKPYKCNLCDHACTQASKL |
| | | KRHMKTHMHKSSPMTVKSDDGLSTASSPEP |
| | | GTSDLVGSASSALKSVVAKFKSENDPNLIP |
| | | ENGDEEEEEDDEEEEEEEEEEEEELTESER |
| | | VDYGFGLSLEAARHHENSSRGAVVGVGDES |
| | | RALPDVMQGMVLSSMQHFSEAFHQVLGEKH |
| | | KRGHLAEAEGHRDTCDEDSVAGESDRIDDG |
| | | TVNGRGCSPGESASGGLSKKLLLGSPSSLS |
| | | PFSKRIKLEKEFDLPPAAMPNTENVYSQWL |
| | | AGYAASRQLKDPFLSFGDSRQSPFASSSEH |
| | | SSENGSLRFSTPPGELDGGISGRSGTGSGG |
| | | STPHISGPGPGRPSSKEGRRSDTCSSHTPI |
| | | RRSTQRAQDVWQFSDGSSRALKF |

While various embodiments have been described above, it should be understood that such disclosures have been presented by way of example only and are not limiting. Thus, the breadth and scope of the subject compositions and methods should not be limited by any of the above-described exemplary embodiments, but should be defined only in accordance with the following claims and their equivalents.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the components and steps in any sequence which is effective to meet the objectives there intended, unless the context specifically indicates the contrary.

What is claimed is:

1. A method of inducing rejuvenation in a population of adult glial progenitor cells, said method comprising:
administering, to the population of adult glial progenitor cells, an effective amount of an expression vector comprising a nucleotide sequence encoding B-cell lymphoma/leukemia 11A (BCL11A) and a regulatory element operably linked to the nucleotide sequence.

2. A method of treating a subject having a glial cell-related disorder, comprising:
administering, to a population of adult glial progenitor cells of the subject, an effective amount of an expression vector comprising a nucleotide sequence encoding B-cell lymphoma/leukemia 11A (BCL11A) and a regulatory element operably linked to the nucleotide sequence.

3. The method of claim 2, wherein the glial cell-related disorder is myelination deficiency selected from the group consisting of multiple sclerosis, neuromyelitis optica, transverse myelitis, optic neuritis, subcortical stroke, diabetic leukoencephalopathy, hypertensive leukoencephalopathy, age-related white matter disease, spinal cord injury, radiation- or chemotherapy induced demyelination, post-infectious and post-vaccinial leukoencephalitis, periventricular leukomalacia, pediatric leukodystrophies, lysosomal storage diseases, congenital dysmyelination, inflammatory demyelination, vascular demyelination, and cerebral palsy.

4. The method of claim 2, wherein the glial cell-related disorder is a neurodegenerative disease selected from the group consisting of Huntington's disease, frontotemporal dementia, Parkinson's disease, multisystem atrophy, and amyotrophic lateral sclerosis.

5. The method of claim 4, wherein the glial cell-related disorder is Huntington's disease.

6. The method of claim 2, wherein the subject is human and wherein the glial cell-related disorder is a neuropsychiatric disorder selected from the group consisting of schizophrenia, autism spectrum disorder, and bipolar disorder.

7. The method of claim 1, wherein the nucleotide sequence encoding BCL11A comprises a nucleotide sequence that encodes human BCL11A of SEQ ID NO:2, or a function variant thereof.

8. The method of claim 1, wherein the expression vector is a non-viral expression vector.

9. The method of claim 1, wherein the agent is a viral expression vector.

10. The method of claim 9, wherein the viral expression vector is a lentiviral vector.

11. The method of claim 9, wherein the viral expression vector is an AAV vector.

12. The method of claim 1, wherein the regulatory element is a glial cell-specific promoter.

13. The method of claim 1, wherein the regulatory element is an inducible promoter.

14. The method of claim 13, wherein the inducible promoter is a tet-on or tet-off promoter.

15. The method of claim 1, further comprising the step of expressing, in the population of adult glial progenitor cells, one or more genes selected from the group consisting of histone deacetylase 2 (HDAC2), histone-lysine N-methyltransferase EZH2 (EZH2), myc proto-oncogene protein (MYC), high mobility group protein HMGI-C(HMGA2), nuclear factor 1 B-type (NFIB), and transcriptional enhancer factor TEF-4 10 (TEAD2).

16. The method of claim 1, further comprising the step of expressing, in the population of adult glial progenitor cells, one or more glial cell-specific genes selected from the group consisting of PDGFRA, ZNF488, GPR17, OLIG2, CSPG4, and SOX10.

17. An expression vector comprising:
a nucleic acid sequence encoding B-cell lymphoma/leukemia 11A (BCL11A); and
a regulatory element operably linked to the nucleic acid sequence,
wherein the expression vector is a lentiviral vector or AAV vector.

18. The expression vector of claim 17, wherein the regulatory element comprises a glial progenitor cell-specific promoter or an inducible promoter.

19. The expression vector of claim 17, wherein the nucleotide sequence encoding BCL11A comprises a nucleotide sequence that encodes human BCL11A of SEQ ID NO:2, or a function variant thereof.

20. An adult human glial progenitor cell harboring the expression vector of claim 17.


SEQUENCE LISTING (2 sequences)

SEQ ID NO: 1 - DNA, 2319 bp, Homo sapiens
  misc_feature 1..2319: note = Human BCL11A (Variant 2) DNA sequence
  source 1..2319: mol_type = genomic DNA
atgtctcgccgcaagcaaggcaaaccccagcacttaagcaaacgggaattctcgcccgagcctcttgaagccattcttacagatgatgaaccagaccacggcccgttgggagctccagaaggggatcatgacctcctcacctgtgggcagtgccagatgaacttcccattgggggacattcttatttttatcgagcacaaacggaaacaatgcaatggcagcctctgcttagaaaaagctgtggataagccaccttccccttcaccaatcgagatgaaaaaagcatccaatcccgtggaggttggcatccaggtcacgccagaggatgacgattgtttatcaacgtcatctagaggaatttgccccaaacaggaacacatagcagataaacttctgcactggaggggcctctcctcccctcgttctgcacatggagctctaatccccacgcctgggatgagtgcagaatatgccccgcagggtatttgtaaagatgagcccagcagctacacatgtacaacttgcaaacagccattcaccagtgcatggtttctcttgcaacacgcacagaacactcatggattaagaatctacttagaaagcgaacacggaagtcccctgaccccgcgggttggtatcccttcaggactaggtgcagaatgtccttcccagccacctctccatgggattcatattgcagacaataacccctttaacctgctaagaataccaggatcagtatcgagagaggcttccggcctggcagaagggcgctttccacccactccccccctgtttagtccaccaccgagacatcacttggacccccaccgcatagagcgcctgggggcggaagagatggccctggccacccatcacccgagtgcctttgacagggtgctgcggttgaatccaatggctatggagcctcccgccatggatttctctaggagacttagagagctggcagggaacacgtctagcccaccgctgtccccaggccggcccagccctatgcaaaggttactgcaaccattccagccaggtagcaagccgcccttcctggcgacgccccccctccctcctctgcaatccgcccctcctccctcccagcccccggtcaagtccaagtcatgcgagttctgcggcaagacgttcaaatttcagagcaacctggtggtgcaccggcgcagccacacgggcgagaagccctacaagtgcaacctgtgcgaccacgcgtgcacccaggccagcaagctgaagcgccacatgaagacgcacatgcacaaatcgtcccccatgacggtcaagtccgacgacggtctctccaccgccagctccccggaacccggcaccagcgacttggtgggcagcgccagcagcgcgctcaagtccgtggtggccaagttcaagagcgagaacgaccccaacctgatcccggagaacggggacgaggaggaagaggaggacgacgaggaagaggaagaagaggaggaagaggaggaggaggagctgacggagagcgagagggtggactacggcttcgggctgagcctggaggcggcgcgccaccacgagaacagctcgcggggcgcggtcgtgggcgtgggcgacgagagccgcgccctgcccgacgtcatgcagggcatggtgctcagctccatgcagcacttcagcgaggccttccaccaggtcctgggcgagaagcataagcgcggccacctggccgaggccgagggccacagggacacttgcgacgaagactcggtggccggcgagtcggaccgcatagacgatggcactgttaatggccgcggctgctccccgggcgagtcggcctcggggggcctgtccaaaaagctgctgctgggcagccccagctcgctgagccccttctctaagcgcatcaagctcgagaaggagttcgacctgcccccggccgcgatgcccaacacggagaacgtgtactcgcagtggctcgccggctacgcggcctccaggcagctcaaagatcccttccttagcttcggagactccagacaatcgccttttgcctcctcgtcggagcactcctcggagaacgggagcttgcgcttctccacaccgcccggggagctggacggagggatctcggggcgcagcggcacgggaagtggagggagcacgccccatattagtggtccgggcccgggcaggcccagctcaaaagagggcagacgcagcgacacttgttcttcacacacccccattcggcgtagtacccagagagctcaagatgtgtggcagttttcggatggaagctcgagagcccttaagttc

SEQ ID NO: 2 - protein, 773 aa, Homo sapiens
  REGION 1..773: note = misc_feature - Human BCL11A (Variant 2) amino acid sequence
MSRRKQGKPQHLSKREFSPEPLEAILTDDEPDHGPLGAPEGDHDLLTCGQCQMNFPLGDILIFIEHKRKQCNGSLCLEKAVDKPPSPSPIEMKKASNPVEVGIQVTPEDDDCLSTSSRGICPKQEHIADKLLHWRGLSSPRSAHGALIPTPGMSAEYAPQGICKDEPSSYTCTTCKQPFTSAWFLLQHAQNTHGLRIYLESEHGSPLTPRVGIPSGLGAECPSQPPLHGIHIADNNPFNLLRIPGSVSREASGLAEGRFPPTPPLFSPPPRHHLDPHRIERLGAEEMALATHHPSAFDRVLRLNPMAMEPPAMDFSRRLRELAGNTSSPPLSPGRPSPMQRLLQPFQPGSKPPFLATPPLPPLQSAPPPSQPPVKSKSCEFCGKTFKFQSNLVVHRRSHTGEKPYKCNLCDHACTQASKLKRHMKTHMHKSSPMTVKSDDGLSTASSPEPGTSDLVGSASSALKSVVAKFKSENDPNLIPENGDEEEEEDDEEEEEEEEEEEEELTESERVDYGFGLSLEAARHHENSSRGAVVGVGDESRALPDVMQGMVLSSMQHFSEAFHQVLGEKHKRGHLAEAEGHRDTCDEDSVAGESDRIDDGTVNGRGCSPGESASGGLSKKLLLGSPSSLSPFSKRIKLEKEFDLPPAAMPNTENVYSQWLAGYAASRQLKDPFLSFGDSRQSPFASSSEHSSENGSLRFSTPPGELDGGISGRSGTGSGGSTPHISGPGPGRPSSKEGRRSDTCSSHTPIRRSTQRAQDVWQFSDGSSRALKF